(19) United States
(12) Patent Application Publication
(10) Pub. No.: US 2021/0348119 A1
(43) Pub. Date: Nov. 11, 2021

(54) COMPOSITIONS AND METHODS FOR CELL CULTURE

(71) Applicant: The United States of America as Represented By The Secretary of the Departmen of Health and Human, Rockville, MD (US)

(72) Inventors: Ilyas SINGEC, Rockville, MD (US); Yu CHEN, Rockville, MD (US); Anton SIMEONOV, Rockville, MD (US)

(73) Assignee: The United States of America as Represented By The Secretary of The Department of Health and Human, Rockville, MD (US)

(21) Appl. No.: 17/284,215

(22) PCT Filed: Oct. 11, 2019
(86) PCT No.: PCT/US2019/055940

§ 371 Date: Apr. 9, 2021

Related U.S. Application Data
(60) Provisional application No. 62/744,395, filed on Oct. 11, 2018.

(51) Int. Cl. C12N 5/00

(52) U.S. Cl. CPC C12N 5/0018; C12N 2501/999

(57) ABSTRACT

Compositions and methods useful for culturing mammalian cells, tissues and/or organs are provided. The compositions contain Chroman 1 or its derivatives, may further include Emricasan or its derivatives, and may further include one or both of trans-ISRIB and polyamines. The compositions may be formulated as media additives, media concentrates or working media. The compositions and methods may be used to culture stem cells, progenitor cells, differentiated cells, isolated primary cells, secondary cells, immortalized cells, cell line cells, germline cells, somatic cells, or modified cells, including genetically modified cells. The compositions and methods may be used to culture, preserve or maintain cells, tissues, organs parts or organs, grow embryoid bodies, embryos, organs or organ parts.

### PRIOR RELATED APPLICATION

The present application claims the benefit of priority of U.S. Provisional Application No. 62/744,395, filed Oct. 11, 2018, which is incorporated herein by reference in its entirety.

### GOVERNMENT LICENSE RIGHTS

This invention was made with Government support awarded by NIH Regenerative Medicine Program of the National Institutes of Health (NIH Common Fund), National Center for Advancing Translational Sciences (NCATS). The Government has certain rights in the invention.

### FIELD

The present invention lies in the fields of biochemistry, cell biology, bioengineering, stem cell biology, regenerative medicine, as well as the related fields, and concerns the compositions and the methods useful for culturing mammalian cells, including, but not limited to, pluripotent stem cells, tissues and organs.

### BACKGROUND

Pluripotency is a remarkable cellular state that allows differentiation of stem cells into any cell type of the human body. Pluripotent stem cells hold enormous potential for drug discovery, disease modeling, and regenerative medicine, especially with the advent of the induced pluripotent stem cell (iPSC) technology. iPSC technology involves reprogramming of non-pluripotent cells into pluripotent cells, thus allowing artificial generation of pluripotent cells from any individual. The resulting iPSCs are attractive for therapeutic and research purposes, as they can be generated from donor cells with desired genetic and/or immunological backgrounds. To fully utilize the therapeutic and research potential of iPSCs, their cell culture conditions require chemically defined media and well-characterized reagents. iPSC culture conditions should also be adaptable to large-scale preclinical and clinical applications. Some examples of such applications are cell replacement therapy, gene therapy and genome editing.

Since the establishment of the first embryonic stem cell (ESC) lines, pluripotent stem cells were known to be highly sensitive to cell dissociation and routine passaging. In comparison to other types of cell culture, culturing of pluripotent stem cells requires particular attention to the culture conditions in order to maintain reasonably high cell survival rates and to maintain the undifferentiated state of pluripotent stem cells over extended periods of time. Human pluripotent stem cells are particularly sensitive to cell culture conditions, especially when the cells are enzymatically dissociated into single cells and plated at very low cell density or in a single-cell-per-well condition required for clonal analysis. Currently available options for single cell cloning have poor outcomes and are labor-intensive. Improving the outcomes of single cell cloning is important for a variety of applications, such as genome editing of iPSCs for preclinical research and clinical applications, for example, correcting a genetic defect through personalized cell therapy, introducing a genetic mutation for disease modeling or introducing a transgene to generate a reporter cell line for drug discovery.

Several chemical compounds have been used as cell-culture media supplements in an attempt to improve the outcomes of stem cell culturing. For example, Rho-associated protein kinase (ROCK) inhibitor termed Y-27632 improves survival of pluripotent stem cells in culture. Some other compounds, such as blebbistatin, thiazovivin, pyrintegrin and pinacidil, were also used, but Y-27632 is still the most widely used reagent in the stem cell field. A variety of cell passaging methods, such as mechanical passaging with special tools, enzymatic passaging with non-trypsin proteases, enzyme-free passaging with ethylenediaminetetraacetic acid (EDTA), have been developed with the goal of reducing cellular stress during cell culture. In spite of many developments in the field, the problems that arise during the culturing of pluripotent stem cells have not yet been resolved. Pluripotent stem cell culture remained challenging, labor-intensive, prone to human error and difficult to reproduce. While there is a need for improved processes and compositions for culturing pluripotent stem cells, such processes and compositions may also be adapted to improve culturing of other mammalian cells, tissues and organs. For example, many widely used cancer cell lines have been maintained for many years in culture acquiring genetic changes over time, these cell lines likely do not faithfully represent in vivo cancer cell biology. At the same time, establishing cell lines from primary tumor can be a daunting task due to poor cell survival in vitro. Accordingly, there is a need for improved processes and compositions for establishing new cancer cell lines from primary tumors.

### SUMMARY

Described herein and included among the embodiments of the present invention are methods, compositions and kits useful for growth and maintenance in culture of mammalian cells, tissues and/or organs. Pluripotent stem cells are highly sensitive to cell culture conditions and undergo apoptosis during routine passaging with and without enzymatic cell dissociation, cryopreservation/thawing, and when prompted to differentiate in two- and three-dimensional cultures. The inventors discovered that certain small chemical compounds, among which are Chroman 1, Caspase-3 inhibitors such as Emricasan, trans-ISRIB and polyamines, and/or the combinations of such compounds, significantly improved pluripotent stem cell survival in culture. In connection with this discovery, the inventors conceived the compositions, kits and methods described herein, which can be used to improve the outcomes of culturing not only of pluripotent stem cells, but also of a variety of other mammalian cells, tissues or organs. Thus, various embodiments of the methods described herein utilize the compositions and/or kits conceived by the inventors for culturing mammalian cells (including but not limited to, pluripotent stem cells) tissues and/or organs. Among other things, the compositions, kits and methods described in this document allow for economical culture process development, cost-effective establishment of new cell lines, scalability of cell culture for drug and/or toxicology screening, robustness and standardization of cell expansion, significantly improved reproducibility of cell differentiation protocols, as well as improved applications that involve cell culture, such as gene therapy and genome editing for personalized medicine. The advantages of the compositions, kits and methods of the present invention are discussed throughout this document and illustrated in the accompanying figures.

The terms “invention,” “the invention,” “this invention” and “the present invention,” as used in this document, are intended to refer broadly to all of the subject matter of this patent application and the claims below. Statements containing these terms should be understood not to limit the subject matter described herein or to limit the meaning or scope of the patent claims below. Covered embodiments of the invention are defined by the claims, not this summary. This summary is a high-level overview of various aspects of the invention and introduces some of the concepts that are described and illustrated in the present document and the accompanying figures. This summary is not intended to identify key or essential features of the claimed subject matter, nor is it intended to be used in isolation to determine the scope of the claimed subject matter. The subject matter should be understood by reference to appropriate portions of the entire specification, any or all figures and each claim. The present document describes and refers to various embodiments of the invention. No particular embodiment is intended to define the scope of the invention. Rather, the embodiments merely provide non-limiting examples of various methods, compositions, kits, systems etc. that are at least included within the scope of the invention. Some embodiments of the present invention are summarized below, while others are described and shown elsewhere in the present document.

Exemplary embodiments of the present invention include compositions comprising Chroman 1 and/or a derivative thereof and one or more caspase-3 inhibitors such as Emricasan and/or a derivative thereof. The compositions can further comprise one or both of trans-ISRIB and polyamines. Embodiments of the compositions can be formulated to provide a concentration of Chroman 1 and/or the derivative thereof of about 4 nM to about 80 μM, about 4 nM to about 40 μM, about 10 nM to about 20 μM, about 20 nM to about 10 μM or about 30 nM to about 500 nM, when incorporated into a medium. Embodiments of the compositions can be formulated to provide a concentration of Emricasan and/or the derivative thereof of about 100 nM to about 80 μM, about 100 nM to about 40 μM, about 200 nM to about 30 μM, about 300 nM to about 20 μM, when incorporated into the medium. Embodiments of the compositions can be formulated to provide a concentration of trans-ISRIB of about 50 nM to about 80 μM, about 50 nM to about 6.25 μM, about 100 nM to about 6.25 μM, or about 200 nM to about 6.25 μM, when incorporated into the medium. Embodiments of the compositions can comprise polyamines comprising one or more of spermine, spermidine, and putrescine, and such embodiments can be formulated to provide a concentration of polyamines of about 0.5 μM to 1 mM, when incorporated into the medium. Embodiments of the compositions can comprise polyamines comprising one or more of spermine, spermidine and putrescine, and such embodiments can be formulated to provide a concentration of spermine of about 0.5 nM to 1 mM, and/or a concentration of spermidine of about 0.5 nM to 1 mM, and/or a concentration of putrescine of about 0.5 nM to 1 mM, when incorporated into the medium.

Exemplary embodiments of the present invention include medium, for example, culture medium compositions comprising components configured to support at least one mammalian cell in vitro or ex vivo and Chroman 1 and/or a derivative thereof. For example, embodiments of the medium compositions can comprise Chroman 1 and/or the derivative thereof at an effective concentration of about 4 nM to 80 μM, 4 nM to 40 μM, 10 nM to 20 μM, 20 nM to 10 μM or 30 nM to 500 nM, wherein the effective concentration of Chroman 1 and/or the derivative thereof is a concentration of Chroman 1 and/or the derivative thereof in a working medium to be used without further dilution. Embodiments of the medium compositions can further include Emricasan and/or a derivative thereof. For example, embodiments of the medium compositions can comprise Emricasan at an effective concentration of about 100 nM to 80 μM, 100 nM to 40 μM, 200 nM to 300 μM, 300 nM to 20 μM, wherein the effective concentration of Emricasan and/or the derivative thereof is a concentration Emricasan and/or the derivative in the working medium to be used without further dilution. Embodiments of the medium compositions can further comprise trans-ISRIB. For example, embodiments of the medium compositions can comprise trans-ISRIB at an effective concentration of about 50 nM to 80 μM, 50 nM to 6.25 μM, 100 nM to 6.25 μM, or 200 nM to 6.25 μM, wherein the effective concentration of trans-ISRIB is trans-ISRIB concentration in the working medium to be used without further dilution. Embodiments of the medium compositions can further comprise polyamines comprising one or more of spermine, spermidine and putrescine. Such embodiments of the medium compositions can comprise an effective concentration of spermine of about 0.5 nM to 1 mM, an effective concentration of spermidine of about 0.5 nM to 1 mM, an effective concentration of putrescine of about 0.5 nM to 1 mM, and wherein the effective concentration of each of spermine, spermidine and/or putrescine is a concentration in the working medium to be used without further dilution. In the embodiments of the medium compositions, the components configured to support the at least one mammalian cell in vitro or ex vivo can comprise one or more of a buffer, an inorganic salt, essential amino acids, a carbohydrate, fatty acids, lipids, vitamins and trace elements. Embodiments of the medium compositions encompass liquid or solid culture media concentrates formulated to be dissolved prior to being used. Embodiments of the medium compositions also encompass media, such as culture media, formulated to be used without further dilution. Embodiments of the medium compositions encompass liquid, semi-solid and solid media. Embodiments of the medium compositions include defined culture media and undefined culture media. Embodiments of the media compositions can be configured for culturing or preservation of one or more of: embryonic stem cells, non-embryonic stem cells, pluripotent stem cells, induced pluripotent stem cells, multipotent stem cells, adult stem cells, progenitor cells, differentiated cells, isolated primary cells, secondary cells, immortalized cells, cell line cells, germline cells, somatic cells, modified cells (including genetically modified cells), human cells or human-derived cells. Embodiments of the culture media compositions can be configured for culturing or preservation of one or more of: plurality of cells, cell cultures, tissue cultures, tissues, organs, organ parts, blastoderms, embryoid bodies or embryos.

Exemplary embodiments of the present invention include kits comprising the composition according to the embodiments of the present invention and medium, such as culture medium components configured to support at least one mammalian cell in vitro or ex vivo. In some exemplary embodiments, such kits can further comprise one or more of a culture vessel, a support or a scaffold for growth of the at least one mammalian cell in vitro or ex vivo. In some other exemplary embodiments, such kits can further comprise one or more of a vessel and other components for preservation of the at least one mammalian cell in vitro or ex vivo.

Exemplary embodiments of the present invention also include compositions comprising the at least one mammalian cell and a medium composition according to one or more of the embodiments of the present invention. The at least one mammalian cell included in such embodiments can be one or more of an embryonic stem cell, a non-embryonic stem cell, a pluripotent stem cell, an induced pluripotent stem cell, a multipotent stem cell, an adult stem cell, a progenitor cell, a differentiated cell, an isolated primary cell, a secondary cell, an immortalized cell, a cell line cell, a germline cell, a somatic cell or a modified cell (including a genetically modified cell). The at least one mammalian cell can also be one or more of a plurality of cells, a cell culture, a tissue culture, a tissue, an organ, an organ part, a blastoderm, an embryoid body or an embryo. The at least one mammalian cell can be a human cell and/or a human-derived cell. The at least one mammalian cell can be thawed. The embodiments of such compositions can comprise a vessel containing the medium. The embodiments of such compositions can comprise solid support for the at least one mammalian cell and/or a scaffold.

Exemplary embodiments of the present invention also include various methods. For example, included among the embodiments of the present invention is a method of preparing a medium according to one or more of the embodiments of the present invention. In another example, included among the embodiments of the present invention is a method of culturing at least one mammalian cell, comprising incubating the at least one mammalian cell in vitro or ex vivo a medium according to one or more of the embodiments of the present invention. In one more example, included among the embodiments of the present invention is a method of obtaining a clonal population of mammalian cells, comprising incubating a dissociated mammalian cell in a culture medium according to one or more of the embodiments of the present invention, until a colony of cells is established from the dissociated mammalian cell. In yet one more example, included among the embodiments of the present invention is a method of obtaining an embryoid body, comprising incubating one or more mammalian cell in a culture medium according to one or more of the embodiments of the present invention until the embryoid body is established. In yet one more example, included among the embodiments of the present invention is a method of growing at least a part of organ in vitro or ex vivo, comprising incubating one or more mammalian cell in the culture medium according to one or more of the embodiments of the present invention until the at least the part of the organ is established. In the above methods, the culture medium according to one or more of the embodiments of the present invention can be a liquid, a semi-solid or a solid medium. The culture medium can be a defined culture medium or an undefined culture medium. Also included among the embodiments of the present invention are methods of maintaining or preserving a cell, a plurality of cells, a tissue, an embryoid body, an embryo, an organoid or at least a part of organ in vitro or ex vivo, comprising incubating one or more of a cell, a plurality of cells, a tissue, an embryoid body, an embryo, an organoid or at least a part of organ in a medium according to one or more of the embodiments of the present invention. The above methods of maintaining or preserving can be performed at various temperatures, including sub-zero temperatures (as is done for cryopreservation). The above methods can be performed on, as appropriate, one or more of: embryonic stem cells, non-embryonic stem cells, pluripotent stem cells, induced pluripotent stem cells, multipotent stem cells, adult stem cells, progenitor cells, differentiated cells, isolated primary cells, secondary cells, immortalized cells, cell line cells, germline cells, somatic cells or modified cells (including genetically modified cells), human cells or human derived cells. The above methods related to culture, growth, maintenance or preservation of live cells can be performed on, as appropriate, one or more of: pluralities of cells, cell cultures, tissue cultures, tissues, organs, organ parts, blastoderms, embryoid bodies or embryos. The above methods can be performed on thawed cells, cell cultures, tissue cultures, tissues, organs, organ parts, blastoderms, embryoid bodies or embryos.

### BRIEF DESCRIPTION OF THE FIGURES

FIG. 1 depicts structures of small molecules.

FIG. 2 is a schematic illustration of the procedure used for quantitative high-throughput screening (qHTS).

FIG. 3 is a scatter plot of maximum survival achieved by the screened compounds (plotted on the X-axis). The survival values plotted on the Y-axis were normalized based on the survival achieved at 10 μM Y-27632 (taken to be 100%).

FIG. 4 shows line plots of dose-response curves of selected ROCK inhibitors Chroman 1, Fasudil HCL, Thiazovivin and Y-27632. Molar concentrations of the selected ROCK inhibitors are plotted on the X-axis (log scale). Four replicates were tested for each concentration and the data were normalized with respect to the average CellTiter Glow™ (CTG) reading obtained from 10 μM Y-27632 Thusly normalized data are plotted on the Y-axis.

FIG. 5 shows a bar graph illustrating the comparison of the effects of Y-27632 and Chroman 1 on the survival of dissociated H9 cells (reduced number of propidium iodide-positive dead cells) seeded into vitronectin coated 6-well plates.

FIG. 6 is a line plot illustrating half maximal inhibitory concentration (IC₅₀) of Y-27632 determined in kinase assays against ROCK1 and ROCK2 using the HotSpot Kinase Assay performed by Reaction Biology Corporation (Malvern, Pa.).

FIG. 7 is a line plot illustrating the half maximal inhibitory concentration (IC₅₀) of Chroman 1 determined in kinase assays against ROCK1 and ROCK2 using the HotSpot Kinase Assay performed by Reaction Biology Corporation (Malvern, Pa.).

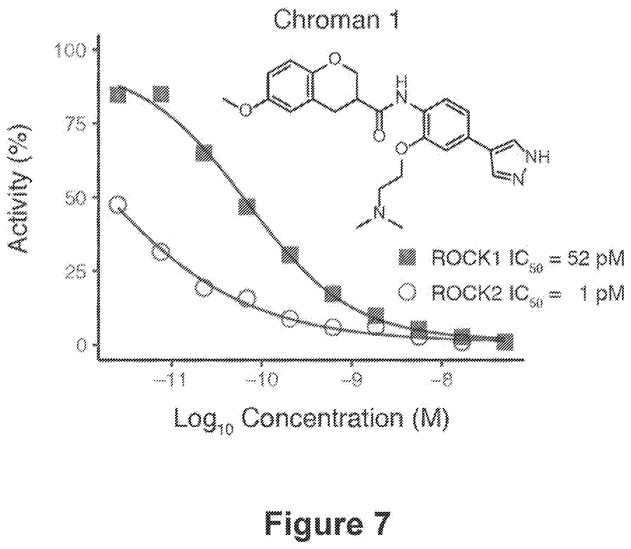

FIG. 8 is a table summarizing the inhibitory activity of 10 μM Y-27632 and 50 nM Chroman 1.

FIG. 9 shows the results of combinatorial matrix screening for the combination of Chroman 1 and caspase inhibitor Emricasan and the combination of Chroman 1 and (−)-Blebbistatin.

FIG. 10 shows a bar graph illustrating improved cell survival when Chroman 1 and Emricasan were combined. CellTiter-Glo® assay was used to quantify viable H9 cells 24 hours post-plating (100,000 cells/cm²).

FIG. 11 shows phase-contrast microscopy images of a time-lapse experiments monitoring cell behavior over 24 hours (IncuCyte Zoom™ Live Cell Analysis, Sartorius, DE).

FIG. 12 is a bar graph illustrating the validation of hits from the primary screening of dissociated stem cells at low cell density.

FIG. 13 shows bar graphs colony formation rate and colony size of H9 cells when treated with different small molecules and small molecule combinations.

FIG. 14 shows representative microscopic images of the colonies that were obtained with Y-27632 and CEPT as quantified in FIG. 14. Whole-well images (6-well plate) were captured with calcein green (0.5 μg/mL; obtained from Thermo Fisher Scientific).

FIG. 15 shows bar graphs summarizing the results of single-cell cloning experiments performed using H9 cells that were plated as 1 cell/well condition (96-well plates).

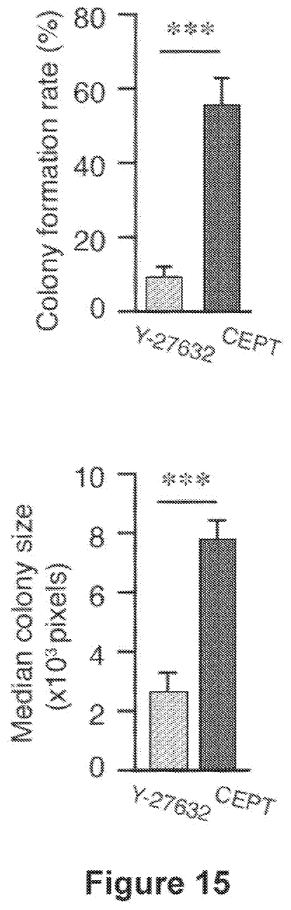

FIG. 16 shows representative microscopic images illustrating the superiority of CEPT for embryoid body formation. The images shown are representative phase contrast images of embryoid bodies from H9 cells. The mages were taken 24 hours after cell plating (20,000 cells/cm²)

FIG. 17 shows a scatter plot illustrating the quantification of the diameter of single embryoid bodies formed from the cells treated with either Y-27632 or CEPT. To generate single embryoid bodies, 1-19 cells were dissociated with Accutase and plated into AggreWell plates (StemCell Technologies, catalog number: 34825) at 5,000 cells/well. For diameter quantification, the images were taken 24 hours post-plating.

FIG. 18 shows representative images illustrating that CEPT improved cerebral organoid formation, as compared to Y-27632.

FIG. 19 is a bar graph illustrating improved thawing of cryopreserved pluripotent stem cells (1-19).

FIG. 20 is a bar graph illustrating CEPT-improved thawing of various iPSC-derived differentiated cells (with the exception of iPSC-derived astrocytes generated by NCATS scientists, all other cells types are commercially available from Fujifilm Cellular Dynamics International).

FIG. 21 illustrates the results of electrophysiological characterization of iPSC-derived cardiomyocytes (commercially available from Fujifilm Cellular Dynamics International) 5 days post-thawing using multi-electrode arrays (Axion Biosystems).

FIG. 22 shows representative microscopic images illustrating CEPT protection of dissociated cells from multiple stress mechanisms. The scale bars shown are 10 μm. Upper panel: Confocal microscopic analysis of the lamin B1-GFP iPSC reporter line (Allen Institute for Cell Science, Seattle, Wash.) displaying dramatic morphological differences in nuclear shape during cell passaging (30 min after plating). Middle panel: OCT4 expressing cells were immunoreactive for γH2AX when exposed to 0.0001% v/v DMSO and Y-27632 (arrowheads) but not when treated with CEPT (3 hours post-plating). Lower panel: Dramatic cytoskeletal differences during cell passaging (3 hours post-plating), as measured by immunocytochemistry against actin and myosin. Stressed cells showed blebbing (white arrowheads) in the presence of 0.0001% v/v DMSO or form prominent actin stress fibers at the colony edge when exposed to Y-27632 (white arrowheads).

FIG. 23 shows an image of a representative Western blot characterizing hESCs (1-19) treated with Y-27632 or CEPT.

FIG. 24 shows an image of a representative Western blot characterizing hESCs (149) treated with Y-27632 or CEPT.

FIG. 25 shows a representative image illustrating the results of puromycin pulse-chase experiment of hESCs (H9) demonstrating that protein synthesis strongly impaired during cell passaging was rescued by CEPT (3 hours post-passage).

FIG. 26 shows a bar graph illustrating that glutathione levels were significantly higher in hESCs (H9) passaged with CEPT as compared to DMSO and Y-27632 (3 hours post-plating).

FIG. 27 shows the results of the experiments illustrating that CEPT improved genome editing efficiency.

FIG. 28 shows a bar graph comparing survival of human pluripotent stem cells in the presence of various reagents and CEPT.

### DESCRIPTION

The embodiments of the present invention were envisioned at least in part based on the discoveries discussed below. Using combinatorial chemical screening and subsequent experimental analysis, the inventors found that a ROCK inhibitor, Chroman 1, is, unexpectedly, significantly more potent and more specific than commonly used Y-27632 and other known ROCK inhibitors, such as blebbistatin, thiazovivin, pinacidil and fasudil, and improved the survival of human pluripotent stem cells (hPSCs) in culture. The inventors also found that Emricasan and Emricasan-related compound Q-VD-OPh, when used in combination with Chroman 1, further improved survival of hPSCs in culture. At the same time, Emricasan alone was not sufficient to improve cell survival. A combination of Chroman 1 and Emricasan (or Q-VD-OPh) unexpectedly exhibited advantageous synergism for improving cell culture outcomes. The useful and advantageous properties of Chroman 1, used alone or in combination with Emricasan were demonstrated during routine cell culture of PSCs over many passages, which indicated that normal karyotype and developmental potential of PSCs were not compromised. Chroman 1 or the combination of Chroman 1 and Emricasan also dramatically and unexpectedly improved cell survival when cryopreserved human PSCs were thawed. Furthermore, superior cell survival in the presence of Chroman 1 or a combination of Chroman 1 and Emricasan was achieved during embryoid body formation. The significant and advantageous effects of Chroman 1 or a combination of Chroman 1 and Emricasan were demonstrated when free-floating embryoid bodies were generated either in bulk culture or in one-embryoid-body-per-well condition. hPSCs in the presence of Chroman 1 or a combination of Chroman 1 and Emricasan underwent minimal cell death and gave rise to higher quality embryoid bodies, as compared to the embryoid bodies grown in Y-27632. The inventors also discovered additional compounds and cell culture conditions leading to further improvement of cell survival in stringent single cell cloning experiments. For example, using trans-ISRIB and/or polyamines together with Chroman 1 or a combination of Chroman 1 and Emricasan improved cell survival during single cell cloning. In addition, the inventors discovered that, in some embodiments, Emricasan could be replaced with other caspase-3 inhibitors.

Based on the above-summarized experimental studies, it was envisioned that individual compounds or various combinations of compounds (which can be referred to, depending on the context, as “active agent” or “active agents”) described in the “Small Molecules” section of this document are useful for improving the survival of mammalian cells in culture and other related processes. Accordingly, various compositions and related processes and kits were envisioned by the inventors. In one non-limiting example, the inventors envisioned active agents and related processes and kits useful and advantageous for culturing mammalian cells, including, but not limited, PSCs, such as hPSCs, that can be employed in various research and clinical applications, such as, but not limited to, cell reprogramming protocols, establishment of new iPSC lines and genome editing using various methods, for example, the methods using CRISPR/Cas9, transcription activator-like effector nucleases (TALENs) or Zinc-finger nucleases (ZFNs). In another non-limiting example, the inventors envisioned active agents and related processes and kits useful for culturing differentiated cells, such as, but not limited to, commercially available neurons, liver cells, cardiomyocytes or immune-competent T cells relevant for chimeric antigen receptor (CAR) T cell therapy. In yet another non-limiting example, the inventors envisioned active agents and related processes and kits for establishing new cell lines, such as, but not limited to, new cell lines from newly isolated primary cells, cell lines of modified cells (for example, genetically modified CAR T cells useful for immunotherapy) and cell lines of cancer cells useful for research applications.

Various compositions and methods described herein can be useful for, but not limited to, establishing new cell lines from primary cells, including cancer and non-cancer cell lines, reprogramming and establishing new iPSCs, improved culturing of non-stem and stem cells (such as ESCs, iPSCs, adult stem cells including neural stem cells, hematopoietic stem cells, mesenchymal stem cells and other organ-derived stem cells or artificially modified cells), improving cryopreservation and thawing of cells, improving growth and/or differentiation of cells in two- and three-dimensional cultures (for example, neurospheres or organoids), improved organ and tissue preservation (for example, but not limited to, preservation of organs and tissues ex vivo prior to transplantation) and improving genome editing and clone selection of cultured cells in various research and clinical techniques.

### Terms and Concepts

A number of terms and concepts are discussed below. They are intended to facilitate the understanding of various embodiments of the invention in conjunction with the rest of the present document and the accompanying figures. These terms and concepts may be further clarified and understood based on the accepted conventions in the fields of the present invention and the description provided throughout the present document and/or the accompanying figures. Some other terms can be explicitly or implicitly defined in other sections of this document and in the accompanying figures and may be used and understood based on the accepted conventions in the fields of the present invention, the description provided throughout the present document and/or the accompanying figures. The terms not explicitly defined can also be defined and understood based on the accepted conventions in the fields of the present invention and interpreted in the context of the present document and/or the accompanying figures.

As used herein, the terms “a,” “an,” and “the” can refer to “one,” “one or more” or “at least one,” unless specifically noted otherwise.

The term “about” is used herein to indicate that a value includes the inherent variation of error for the device, the method being employed to determine the value, or the variation that exists among the study subjects. For example, the term “about” may mean ±1%, ±5%, ±10%, ±15% or ±20% variation from a predetermined value.

As used herein, the terms “isolate,” “separate” or “purify” and the related terms are not used necessarily to refer to the removal of all materials other than the components of interest from a sample. Instead, in some embodiments, the terms are used to refer to a procedure that enriches the amount of one or more components of interest relative to one or more other components present in the sample. In some embodiments, “isolation,” “separation” or “purification” may be used to remove or decrease the amount of one or more components from a sample. For example, the expression “an isolated cell” can refer to a cell that has been substantially separated or purified away from other cells of a cell culture or an organism.

The expression “derived from” and the related expressions referring to cells or a biological sample indicate that the cell or sample was obtained from the stated source at some point in time. For example, a cell derived from an organism can represent a primary cell obtained directly from the individual (that is, unmodified), or it can be modified, for example, by introduction of a recombinant vector, by exposure to or culturing under particular conditions, or immortalization. In some cases, a cell derived from a given source will undergo cell division and/or differentiation such that the original cell is no longer exists, but the continuing cells will be understood to derive from the same source.

The terms “culture,” “cell culture” and related terms can be used to refer to a cell or a population of cells residing outside of an organism. These cells can be stem cells, primary cells isolated from an organism or obtained from a cell bank, animal, or blood bank, or secondary cells that are derived from such sources. Secondary cells can be immortalized for long-lived cell culture.

A primary cell includes any cell of an adult or fetal organism apart from egg cells, sperm cells and stem cells. Examples of useful primary cells include, but are not limited to, skin cells, bone cells, blood cells, cells of internal organs and cells of connective tissue. A secondary cell is derived from a primary cell and can be immortalized for long-lived in vitro cell culture.

The terms “culture,” “culturing,” “grow,” “growing,” “maintain,” “maintaining,” “expand,” “expanding,” etc., when referring to cell, tissue or organ culture or the process of culturing, can be used interchangeably to mean that a cell or a group of cells (the scope of which expression includes groups or pluralities of undifferentiated or differentiated cells, embryos, embryoid bodes, tissues or organs) is maintained outside the body (ex vivo and/or in vitro) under conditions suitable for survival, proliferation, differentiation and/or avoiding senescence. In other words, cultured cell or groups of cells are allowed to survive, and culturing can result in cell growth, differentiation, or division. The terms above not imply that all cells in the culture survive or grow or divide, as some may naturally senesce. Cells are typically cultured in media, which can be changed during the course of the culture. The so-called two-dimensional (2D) cell cultures grow on flat surfaces, typically in plastic vessels that can be coated with substrates (for example, vitronectin, laminin 521, Matrigel). Three-dimensional (3D) cultures are cultures in which biological cells are permitted to grow or interact with their surroundings in all three dimensions. 3D cultures can be grown in in a variety of artificial environments, such as, but not limited to, plates, flasks, bioreactors or small capsules in which the cells can grow into spheroids. 3D cultures include so-called scaffold-free and scaffold-based technologies. Scaffold-free methods employ, but are not limited to, the uses of low adhesion plates, hanging drop plates, micropatterned surfaces, and rotating bioreactors, magnetic levitation, and magnetic 3D bioprinting. Scaffolds are structures or materials that provide a structural support for cell attachment and, in some cases, differentiation. Scaffolds include solid scaffolds, sponges (such as cellulose sponges), protein-based scaffolds (such as collagen or gelatin-based scaffolds), hydrogels, nanofiber scaffolds, synthetic polymer scaffolds (for example, polycaprolactone or polystyrene scaffolds). In general, the culture environment includes consideration of such factors as the substrate for cell growth, cell density and cell contract, the gas phase, the medium, and temperature. Cells in culture are generally maintained under conditions known to be optimal for cell growth. Such conditions may include, for example, a temperature of approximately 37° C. and a humidified atmosphere containing approximately 5% CO₂. The duration of the incubation can vary widely, depending on the desired results.

The terms “medium,” “culture medium,” “culture solution,” “growth medium” and the related terms and expression refer to a medium supporting the survival and/or growth of cells (including single cells and pluralities of cells), tissues, organs or parts thereof or embryonic structures (such as, but not limited to, morula, blastocoel, blastocyst or embryo). The term “medium” encompasses various types of media, included, but not limited to, media used for cell, tissue or organ culture and media used for cell, tissue and organ preservation. For example, the term “medium” encompasses the media used for cell propagation and/or differentiation, media used for cell, tissue, organoid, organ or embryo growth, as well as media used for cell, tissue, organoid, organ or embryo preservation, including cryopreservation and preservation of cells, tissues or organs ex vivo prior to implantation. A medium is typically isotonic, and can be a liquid, a colloidal liquid, a gel, a solid and/or a semi-solid. A medium can be configured to provide a matrix for cell adhesion or support, or a separate support (such as a culture vessel surface or a scaffold) can be provided. A medium can include the components for nutritional, chemical, and structural support necessary for culturing a cell or cells. A chemically defined medium (or “defined medium”) is a medium with known concentrations of all of its chemical components. In contrast, an undefined medium can contain complex biological components, such as serum albumin or serum, that do not have completely defined compositions. A conditioned medium is understood to be a previously used medium from cultured cells. It contains metabolites, growth factors, and extracellular matrix proteins secreted into the medium by the cultured cells, which can be beneficial for subsequent uses of such conditioned medium.

The expression “single cell cloning” refers to a process that allows generating a monoclonal cell line from a polyclonal pool of cells. Single cell cloning typically involves isolation of individual cells by various approaches, such as single-cell sorting, isolation with cloning cylinders or limiting dilution. Thusly isolated cells are then grown in culture.

In the context of cell culture, the term “dissociating” can refer to a process of isolating cells from other cells or from a surface, such as a culture plate surface. For example, cells can be dissociated from an organ or a tissue by mechanical or enzymatic methods. In another example, cells that aggregate ill vitro can be dissociated from each other. In yet another example, adherent cells are dissociated from a culture plate or other surface. Dissociation can involve breaking cell interactions with extracellular matrix (ECM) and substrates (for example, culture surfaces) or breaking the ECM between cells.

A “stem cell” is a cell characterized by the ability of self-renewal through mitotic cell division and the potential to differentiate into a tissue or an organ. Among stem cells, embryonic and somatic stem cells may be distinguished. For example, mammalian embryonic stem cells may reside in the blastocyst and give rise to embryonic tissues, whereas somatic stem cells may reside in adult tissues for the purpose of tissue regeneration and repair.

The term “cell line” typically refers to a cell culture developed from a single cell of a multicellular organism. Cells of a cell line have a relatively uniform genetic makeup. Some cell lines originate from stem cells. Some cell lines originate from naturally occurring cancerous cells that underwent genetic modifications (such as one or more mutations or introductions of viral genes) leading to uncontrolled proliferation. Some cell lines originate from the cells that have been artificially immortalized by various methods.

The term “self-renewal,” when used in reference to cells, describes their ability to divide and generate at least one daughter cell with the self-renewing characteristics of the parent cell, although one or more of other daughter cells may commit to a particular differentiation pathway. For example, a self-renewing hematopoietic stem cell can divide and form one daughter stem cell and another daughter cell committed to differentiation in the myeloid or lymphoid pathway. Non self-renewing cells can still undergo cell division to produce daughter cells, neither of which have the differentiation potential of the parent cell type, but instead generates differentiated daughter cells.

The terms “pluripotent,” “pluripotency” and the related terms and expressions refer to animal cells or cell populations with the ability to give rise to progeny that can undergo differentiation, under appropriate conditions, into cell types that collectively demonstrate characteristics associated with cell lineages from all of the three germinal layers (endoderm, mesoderm, and ectoderm). For example, the expression “pluripotent stem cell characteristics” refers to characteristics of a cell or a cell population that distinguish pluripotent stem cells or their populations from other cells. The ability to give rise to progeny that can undergo differentiation, under the appropriate conditions, into cell types that collectively demonstrate characteristics associated with cell lineages from all of the three germ layers (endoderm, mesoderm, and ectoderm) is a pluripotent stem cell characteristic. Cell morphologies as well as expression or non-expression of certain combinations of molecular markers are also pluripotent stem cell characteristics. Pluripotent stem cells (PSCs) include embryonic stem cells (ESCs) and induced pluripotent stem cells (iPSCs).

The term “stem cell” and the related terms and expressions are used herein to refer to animal cells that are capable of dividing and renewing themselves for long periods, are unspecialized, and can give rise to specialized cell types. Stem cells are capable of dividing and renewing themselves for long periods. Unlike, for example, muscle cells, blood cells, or nerve cells—which do not normally replicate themselves—stem cells may replicate many times or proliferate. If the resulting cells continue to be unspecialized, like the parent stem cells, the cells are said to be capable of long-term self-renewal.

Embryonic stem cells (ESCs) are derived from embryos and, under appropriate conditions, they can remain undifferentiated (unspecialized) in culture. Embryonic stem cell line is a line of ESCs cultured under the conditions that allow proliferation without differentiation for months to years. Under other conditions, for example, if the cells are allowed to clump together to form embryoid bodies, they begin to differentiate spontaneously.

“Embryoid bodies” are rounded collections of cells that can arise from stem cells cultured in suspension. Embryoid bodies contain cell types derived from all three germ layers.

An “adult stem cell,” which can also be termed “somatic stem cell,” is a stem cell found, in an organism, among differentiated cells in a tissue or organ and can differentiate to yield some or all of the specialized cell times in the tissue or organ. Somatic stem cells can be grown in culture. When differentiating into specialized cells, they typically generate intermediate cells called “precursor” or “progenitor” cells. Somatic stem cells and progenitor cells can be described as “multipotent” or “oligopotent,” depending on their degree of potency. Some examples of somatic stem cells are: hematopoietic stem cells that give rise to all the types of blood cells (red blood cells, B lymphocytes, T lymphocytes, natural killer cells, neutrophils, basophils, eosinophils, monocytes and macrophages); mesenchymal stem cells that include bone marrow stromal stem cells and skeletal stem cells and can give rise to bone cells (osteoblasts and osteocytes), cartilage cells (chondrocytes), fat cells (adipocytes), and stromal cells that support blood formation; neural stem cells that can give rise to nerve cells (neurons), astrocytes and oligodendrocytes; epithelial stem cells in the lining of the digestive tract that can give rise to absorptive cells, goblet cells, Paneth cells, and enteroendocrine cells; skin stem cells that occur in the basal layer of the epidermis (and can give rise to keratinocytes) and at the base of hair follicles (and can give rise to both the hair follicle and to the epidermis) A tissue-specific progenitor cell is a cell devoid of self-renewal potential that is committed to differentiate into cells of a specific organ or tissue. Certain somatic stem cell types can differentiate into cell types seen in organs or tissues other than those expected from the somatic stem cell's origin. This phenomenon is called “transdifferentiation.”

The expression “induced pluripotent stem cell” (iPSC) refers to a pluripotent stem cell artificially derived from a non-pluripotent cell. For example, human iPSCs are artificially derived from a human non-pluripotent cell. iPSCs can be derived by introducing products of specific sets of pluripotency-associated genes, or “reprogramming factors,” into a given cell type and/or exposing non-pluripotent cells to particular conditions.

The term “non-pluripotent cells” refer to mammalian cells that are not pluripotent cells. Examples of such cells include differentiated cells, somatic stem cells, as well as progenitor cells. Some non-pluripotent cells maintain a degree of potency, some of the examples being somatic stem cells and progenitor cells.

“Cell potency” describes a cell's ability to differentiate into other cell types. A cell can be designated as a pluripotent cell, a multipotent cell (which can differentiate into several but not all cell types, for example, umbilical cord blood stem cells and mesenchymal stem cells) or an oligopotent cells (having the ability to differentiate into a few cell types, for example, lymphoid cells or vascular stem cells). Under current understanding, potency exists on a continuum. Thusly, the boundaries between the divisions of cells based on potency may be fluid and are not necessarily limiting.

The term “progenitor cell” refers to the cells that are early descendants of stem cells. They can typically differentiate to form one or more kinds of cells but are non-pluripotent. In other words, they are limited with respect to which kinds of cells they can develop into. Progenitor cells can be primary cells obtained from an organism, cells proliferated in culture or cells derived from stem cells.

“Differentiation” is the process by which a less specialized cell becomes a more specialized cell type. For example, early development of a multicellular animal is characterized by the rapid proliferation of embryonic cells, which then differentiate to produce the many specialized types of cells that make up the tissues and organs of the multicellular animal. As cells differentiate, their rate of proliferation usually decreases. Some types of differentiated cells never divide again, but many differentiated cells are able to resume proliferation as required to replace cells that have been lost as a result of injury or cell death. Some cells divide continuously throughout life to replace cells that have a high rate of turnover in adult multi cellular animals. Examples of differentiated cells include, but are not limited to, cells from a tissue selected from bone marrow, skin, skeletal muscle, fat tissue and peripheral blood. Exemplary differentiated cell types include, but are not limited to, fibroblasts, hepatocytes, myoblasts, neurons, osteoblasts, osteoclasts, and lymphocytes.

Cancer cells are cells that are capable of uncontrolled proliferation, which allows them, in an organism, to form solid tumors or flood blood. Cancer cells are typically formed when the genes involved in cell culture division are modified, for example, by a mutation or a viral infection. Such a modification can be natural or artificially induced. In culture, cancer cells can be used to produce cell cultures. For example, isolation of cells from a naturally occurring or induced cancer can generate an immortalized cell line. Some examples of such immortalized cell lines are human HeLa cells, which were obtained from a cervical cancer, and mouse Raw 264.7 cells, which were subjected to mutagenesis and then selected for cells able to undergo division.

The expression “modified cells” and the related terms and expressions encompass all cells that have been or are derived from the cells that have been artificially modified, by any methods, as compared to the original or cells from which they are derived. Modified cells can be produced from primary cells, secondary cells, stem cells, cultured cells and/or other modified cells. Modifications include, but are not limited to, genetic modification or engineering, in which case modified cells can be referred to as “genetically modified” or “genetically engineered.” Genetic modification can be accomplished by various methods that result in incorporation of foreign or heterologous nucleic acids into the cells being modified. Some examples of such methods are transduction by a virus or a viral vector, or transfection of isolated nucleic acids into cells through transient pores in the cell membrane. Other modifications include exposing the source cells to biological and non-biological molecules or factors or culture conditions. Some examples of modified cells are iPSCs, genetically modified cells, including those used for gene therapies (one example being genetically modified immune system cells, such as T cells modified for CAR T-cell therapy; another example being gene-edited cells, such as those modified using CRISPR/Cas9, TALENs or ZFNs.

The term “vessel” refers to a container, dish, plate, flask, bottle, cell culture tube, a bioreactor and the like, which can be used to culture, maintain or grow a cell, group of cells, tissue or organ ex vivo or in vitro. Suitable vessels include, for example, multi-well plates, wells of multi-well plates, dishes, tubes, flasks, bottles and reactors.

The terms “stabilize” and the related terms and expressions used in reference to cells (for example, “stabilizing a cell”) refer to reduction of negative cell responses, such as cell death or senescence. For example, stem cells and other cells can die in response to dissociation, isolation, freezing and/or thawing. In other words, the above conditions can reduce cell viability. Embodiments of the compositions, methods and kits described therein can mitigate the reduction of cell viability and improve cell survival, which can be described as cell stabilization.

### Small Molecules

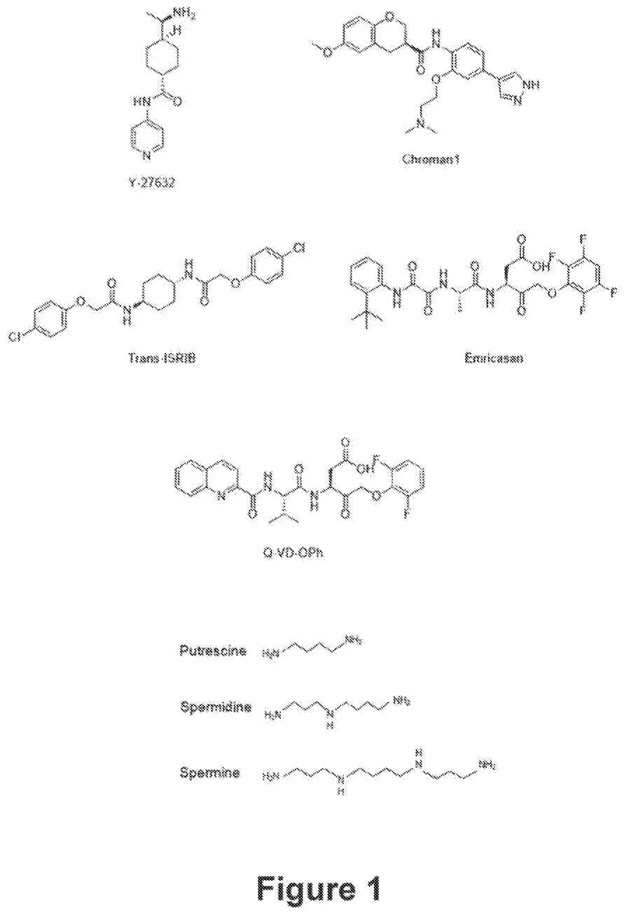

The term “Chroman 1” refers to (3S)—N-{2-[2-(Dimethylamino)ethoxy]-4-(1H-pyrazol-4-yl)phenyl}-6-methoxy-3,4-dihydro-2H-1-benzopyran-3-carboxamide, with the structure shown in FIG. 1. Chroman-related compounds or derivatives are structurally-related compounds (Chroman moiety-containing ROCK inhibitors), some of which are described in Chen et al. “Chroman-3-amides as potent Rho kinase inhibitors” Bioorganic and Medicinal Chemistry Letters 18:6406-6409 (2008) and LoGrasso et al. “Rho Kinase (ROCK) Inhibitors and Their Application to Inflammatory Disorders” Current Topics in Medicinal Chemistry 9:704-723 (2009). Chroman 1, its derivatives or related compounds can be supplied as a salt or in solution.

The term “caspase inhibitor” refers to small molecules that act by binding to the active site of caspases either in a reversible or irreversible manner. They are available as either pan-caspase or caspase-specific inhibitors. In some embodiments of the invention the caspase inhibitor is a caspase-3 inhibitor. Exemplary caspase-3 inhibitors are Emricasan, Z-VAD-FMK (benzyloxycarbonyl-Val-Ala-Asp(OMe)-fluoromethylketone), Z-DQMD-FMK (Z-Asp(OMe)-Gln-Met-Asp(OMe) fluoromethyl ketone), Z-DEVD-FMK (benzyloxycarbonyl-Asp(OMe)-Glu(OMe)-Val-Asp(OMe)-fluoromethylketone) or Ac-DEVD-CHO (N-acetyl-Asp-Glu-Val-Asp aldehyde).

The term “Emricasan” refers to 3-(2-(2-tert-butylphenylaminooxalyl) aminopropionylamino)-4-oxo-5-(2,3,5,6-tetrafluorophenoxy)pentanoic acid, with the structure shown in FIG. 1. Emricasan-related compounds or derivatives are structurally-related compounds (such as Q-VD-OPh hydrate), some of which are described in Linton et al. “First-in-Class Pan Caspase Inhibitor Developed for the Treatment of Liver Disease” . Med. Chem. 48:6779-6782, (2005). Emricasan, its derivatives or related compounds can be supplied as a salt or in solution.

The term “trans-ISRIB,” which can be used interchangeably with the terms “ISRIB” or “ISRIB (trans-isomer)” refers to N,N′-((1r,4r)-cyclohexane-1,4-diyl)bis(2-(4-chlorophenoxy)acetamide) with the structure shown in FIG. 1. As described in Sidrauski et al. “Pharmacological brake-release of mRNA translation enhances cognitive memory” eLIFE 2:e00498 (2013), trans-ISRIB is 100-fold more potent (IC₅₀=5 nM) than cis-ISRIB (IC₅₀=600 nM) suggesting a stereospecific interaction with the cellular target. Trans-ISRIB can be supplied as a salt or in solution.

The term “polyamines”, as used herein, refers to one or more of the polycations putrescine, spermidine and spermine, with the structure shown in FIG. 1, which are known to interact with negatively charged macromolecules, such as DNA, RNA and proteins.

The term “comprising” and the related terms (“comprise,” “comprises,” etc.), when used in this document to describe various embodiments of the invention, are open-ended, meaning that they do not exclude additional elements and synonymous with terms “including,” “containing” or “having.” When an embodiment of the invention is described using the term “comprising,” it is intended to include the embodiments, in which the term comprising is replaced with the terms “consisting of” or “consisting essentially of”.” In other words, the description of the embodiments of the invention described in this document using the term “comprising” and the related terms also provides the description of the related embodiments that use “consisting of” or “consisting essentially of” instead of “comprising”. The term “consisting of” excludes any elements (steps, ingredient etc.) not specified in the description. The term “consisting essentially of” is intended to exclude only those elements not specified in the description that do not materially affect the basic and novel characteristics of the embodiment.

### Compositions

In some embodiments, the present invention provides compositions that can be used for culturing various types of mammalian cells. The compositions can comprise a ROCK inhibitor (a compound that inhibits the activity Rho kinase (ROCK)). For example, a composition according to embodiments of the present invention can comprise Chroman 1 or a related molecule, as described in in the “Small Molecules” section of this document. In another example, a composition according to the embodiments of the present invention can comprise Chroman 1. The compositions can further comprise a caspase inhibitor (a compound inhibiting the activity of one or more cytosolic aspartate-specific cysteine proteases involved in the initiation and execution of apoptosis). A caspase inhibitor can be Emricasan or a related molecule or any caspase inhibitor, as described in the “Small Molecules” section of this document. One example of a composition embodiment comprises a ROCK inhibitor and a caspase inhibitor. Some other examples are compositions comprising Chroman 1 or a related molecule and Emricasan, compositions comprising Chroman 1 and Emricasan or a related molecule, compositions comprising Chroman 1 and Emricasan, compositions comprising a ROCK inhibitor and Emricasan or a related molecule, or compositions comprising a ROCK inhibitor and Emricasan. In addition to a ROCK inhibitor (such as Chroman 1 or a related molecule) or to a ROCK inhibitor and a caspase inhibitor (such as Emricasan or a related molecule), the composition according to the embodiments of the present invention can further comprise one or both of trans-ISRIB and polyamines. In the context of the embodiments of the present invention, each of their components separately or a combination of components, as discussed above, can be referred to as “active agent” or “active agents.”

Various formulations of the compositions according to the embodiments of the present invention are envisioned. For example, some embodiments of the compositions according to the embodiments of the present invention can be formulated as media, for example, culture media, additives and contain amounts of one or more active agents sufficient to provide effective concentrations or effective amounts of the respective active agent or agents upon addition to culture media. In the context of the embodiment of the present invention, effective concentrations or effective amounts are those concentrations or amounts, respectively, of the one or more active agents that elicit desired effects on the cells exposed to the compositions, such as, but not limited to, improved survival (viability), cell stabilization, improved growth, reduced cell death, reduced senescence, improved growth, improved differentiation etc.

For example, a composition according one or more embodiments of the present invention can be formulated to provide a concentration of Chroman 1 (or its active derivative or a related compound), when incorporated into a medium, such as a culture medium, of about 4 nM to about 80 μM, about 10 nM to about 20 μM, about 20 μM to about 10 μM or about 30 nM to about 500 nM, such as about 4 nM, 5 nM, 30 nM, 55 nM, 80 nM, 105 nM, 130 nM, 155 nM, 180 nM, 205 nM, 230 nM, 255 nM, 280 nM, 305 nM, 330 nM, 355 nM, 380 nM, 405 nM, 430 nM, 455 nM, 480 nM, 500 nM. 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 WM, 34 μM, 35 μM, 36 μM, 37 μM, 38 μM, 39 μM, 40 μM, 45 μM, 45 μM, 50 μM, 55 μM, 60 UM, 65 μM, 70 μM, 75 μM or 80 μM. In another example, a composition according to the embodiments of the present invention can be formulated to provide a concentration of Emricasan (or its active derivative or a related compound), when incorporated into a medium, such as a culture medium, of about 5 nM to about 100 μM, about 5 nM to about 80 μM, about 200 nM to about 30 μM, about 300 nM to about 20 μM, for example, about 100 nM, 150 nM, 200 nM, 250 nM, 300 nM, 350 nM, 400 nM, 450 nM, 500 nM, 550 nM, 600 μM, 650 nM, 700 nM, 75011M, 800 nM, 850 nM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 21 μM, 22 μM, 23 μM, 24 μM, 25 μM, 26 μM, 27 μM, 28 nM, 29 μM, 30 μM, 31 nM, 32 μM, 33 μM, 34 μM, 35 μM, 36 nM, 37 μM, 38 μM, 39 μM, 40 μM, 45 μM, 45 μM, 50 μM, 55 μM, 60 μM, 65 μM, 70 μM, 75 nM, 80 μM, 85 μM, 90 μM, 95 μM or 100 μM. In one more example, a composition according to the embodiments of the present invention can be formulated to provide a concentration of trans-ISRIB, when incorporated into a medium, such as a culture medium, of about 5 nM to about 80 μM, about 5 nM to about 50 μM, about 100 nM to about 6.25 nM, or about 200 nM to about 6.25 μM, for example, about 50 nM, 100 μM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 5.25 μM, 5.5 μM, 5.75 WM, 6 μM, 6.25 μM, 6.5 μM, 7 μM, 7.5 μM, 8 μM, 8.5 μM, 9 μM, 9.5 μM, 10 μM, 10.5 μM, 11 nM, 11.5 μM, 12 μM, 12.5 μM, 13 μM, 13.5 μM, 14 μM, 14.5 nM, 15 nM, 15.5 μM, 16 μM, 16.5 μM, 17 nM, 17.5 μM, 18 μM, 18.5 μM, 19 μM, 19.5 μM, 20 μM, 21 μM, 22 μM, 23 nM, 24 nM, 25 μM, 26 nM, 27 μM, 28 μM, 29 μM, 30 μM, 31 μM, 32 μM, 33 μM, 34 μM, 35 μM, 36 μM, 37 nM, 38 μM, 39 μM, 40 μM, 45 nM, 45 μM, 50 μM, 55 μM, 60 μM, 65 μM, 70 μM, 75 μM or 80 μM. In yet one more example, a composition according to the embodiments of the present invention can be formulated to contain polyamines so as to provide a concentration of spermine, when incorporated into medium, such as a culture medium, of about 0.5 nM to 1 mM, for example, about 0.5 nM, 20.5 nM, 40.5 nM, 60.5 nM, 80.5 nM, 100.5 nM, 120.5 nM, 140.5 nM, 160.5 nM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 μM, 20.5 μM, 40.5 μM, 60.5 μM, 80.5 μM, 100.5 nM, 120.5 μM, 140.5 μM, 160.5 μM, 180.5 μM, 200.5 nM, 220.5 μM, 240.5 μM, 260.5 μM, 280.5 μM, 300.5 μM, 320.5 μM, 340.5 μM, 360.5 μM, 380.5 μM, 400.5 μM, 420.5 μM, 440.5 μM, 460.5 μM, 480.5 μM, 500.5 UM, 520.5 UM, 540.5 μM, 560.5 μM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 μM, 860.5 μM, 880.5 μM, 900.5 μM, 920.5 μM, 940.5 μM, 960.5 μM, 980.5 μM or 1 mM. In yet one more example, a composition according to the embodiments of the present invention can be formulated to contain polyamines so as to provide a concentration of spermidine, when incorporated into a medium, such as a culture medium, of about 0.5 μM to 1 mM, for example, approximately 0.5 nM, 20.5 nM, 40.5 nM, 60.5 nM, 80.5 nM, 100.5 nM, 120.5 nM, 140.5 nM, 160.5 μM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 μM, 20.5 μM, 40.5 μM, 60.5 μM, 80.5 μM, 100.5 μM, 120.5 μM, 140.5 μM, 160.5 μM, 180.5 μM, 200.5 μM, 220.5 μM, 240.5 μM, 260.5 μM, 280.5 μM, 300.5 μM, 320.5 μM, 340.5 μM, 360.5 μM, 380.5 μM, 400.5 μM, 420.5 μM, 440.5 μM, 460.5 μM, 480.5 μM, 500.5 μM, 520.5 μM, 540.5 μM, 560.5 μM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 μM, 860.5 μM, 880.5 μM, 900.5 μM, 920.5 μM, 940.5 μM, 960.5 μM, 980.5 μM or 1 mM. In yet one more example, a composition according to the embodiments of the present invention can be formulated to contain polyamines so as to provide a concentration of putrescine, when incorporated into a medium, such as culture medium, of about 0.5 μM to 1 mM, for example, approximately 0.5 nM, 20.5 nM, 40.5 nM, 60.5 nM, 80.5 nM, 100.5 nM, 120.5 nM, 140.5 nM, 160.5 nM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 μM, 20.5 μM, 40.5 μM, 60.5 μM, 80.5 μM, 100.5 μM, 120.5 μM, 140.5 μM, 160.5 μM, 180.5 μM, 200.5 μM, 220.5 μM, 240.5 UM, 260.5 μM, 280.5 μM, 300.5 μM, 320.5 μM, 340.5 μM, 360.5 μM, 380.5 μM, 400.5 μM, 420.5 μM, 440.5 μM, 460.5 μM, 480.5 μM, 500.5 μM, 520.5 μM, 540.5 μM, 560.5 μM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 μM, 860.5 μM, 880.5 μM, 900.5 μM, 920.5 μM, 940.5 μM, 960.5 μM, 980.5 μM or 1 mM.

In addition to one or more active agents, media additives, such as culture media additives, according to the embodiments of the present invention may contain other ingredients, such as, but not limited to, DMEM/F12, ascorbic acid, insulin, selenium, transferrin, NaHCO₃, fibroblast growth factor 2 (FGF-2), transforming growth factor beta (TGF-β). Media additives according to the embodiments of the present invention are formulated so that they can be readily incorporated into media, such as culture media. For example, media additives according to the embodiments of the present invention can be provided in powdered form, as a tablet or as a capsule readily dissolvable in aqueous culture media. In another examples, media additives according to the embodiments of the present invention can be provided as concentrated solutions or as suspensions to be added to media, such as culture media.

Some other embodiments of the compositions according to the embodiments of the present invention can be formulated as a medium, such as culture medium, comprising the above-discussed active agents, and, in addition, components configured to support at least one mammalian cell in vitro or ex vivo. Embodiments of media, such as culture media, can comprise a ROCK inhibitor (a compound that inhibits the activity Rho kinase (ROCK)). For example, a medium, such as culture medium, according to embodiments of the present invention can comprise Chroman 1 or a related molecule, as described in in the “Small Molecules” section of this document. In another example, a medium, such as culture medium, according to the embodiments of the present invention can comprise Chroman 1. The medium, such as culture medium, can further comprise and a caspase inhibitor. A caspase inhibitor can be Emricasan or a related molecule or any other caspase inhibitor, as described in in the “Small Molecules” section of this document. One example of a medium embodiment comprises a ROCK inhibitor and a caspase inhibitor. Some other examples are a medium comprising Chroman 1 or a related molecule and Emricasan, a medium comprising Chroman 1 and Emricasan or a related molecule, a medium comprising Chroman 1 and Emricasan, a medium comprising a ROCK inhibitor and Emricasan or a related molecule, or a medium comprising a ROCK inhibitor and Emricasan. In addition to a ROCK inhibitor (such as Chroman 1 or a related molecule) or to a ROCK inhibitor and a caspase inhibitor (such as Emricasan or a related molecule), the media according to the embodiments of the present invention can further comprise one or both of trans-ISRIB and polyamines.

In a prepared form, media, such as culture media according to the embodiments of the present invention contains effective concentrations or effective amounts of one or more active ingredients. For example, a medium according one or more embodiments of the present invention can contain Chroman 1 (or its active derivative or a related compound) at a concentration of about 4 nM to about 80 μM, about 10 nM to about 20 μM, about 20 nM to about 10 μM or about 30 nM to about 500 nM, such as about 4 nM, 5 nM, 30 nM, 55 nM, 80 nM, 105 nM, 130 nM, 155 nM, 180 nM, 205 nM, 230 nM, 255 nM, 280 nM, 305 nM, 330 nM, 355 nM, 380 nM, 405 nM, 430 nM, 455 nM, 480 nM, 500 nM. 525 nM, 550 nM, 575 nM, 600 nM, 625 nM, 650 nM, 675 nM, 700 nM, 725 nM, 750 nM, 775 nM, 800 nM, 825 nM, 850 nM, 875 nM, 900 nM, 925 nM, 950 nM, 975 nM, 1 μM, 2 μM, 3 μM, 4 μM, 5 μM, 6 μM, 7 μM, 8 μM, 9 μM, 10 μM, 11 μM, 12 μM, 13 μM, 14 μM, 15 μM, 16 μM, 17 μM, 18 μM, 19 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 45 μM, 45 μM, 50 μM, 55 μM, 60 μM, 65 μM, 70 μM, 75 μM or 80 μM. In another example, a medium according to one or more embodiments of the present invention can contain Emricasan (or its active derivative or a related compound) at a concentration of about 5 nM to about 100 μM, about 5 nM to about 80 μM, about 200 nM to about 30 μM, about 300 nM to about 20 μM, for example, about 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 1 μM, 1.5 μM, 2 μM, 2.5 μM, 3 μM, 3.5 μM, 4 μM, 4.5 μM, 5 μM, 5.5 μM, 6 μM, 6.5 μM, 7 μM, 7.5 μM, 8 μM, 8.5 μM, 9 μM, 9.5 μM, 10 μM, 10.5 μM, 11 μM, 11.5 μM, 12 μIVI, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 45 μM, 45 μM, 50 μM, 55 μM, 60 μM, 65 μM, 70 μM, 75 μM, 80 μM, 85 μM, 90 μM, 95 μM or 100 μM. In one more example, a medium according to one or more embodiments of the present invention can contain trans-ISRIB at a concentration of about 5 nM to about 80 μM, about 5 nM to about 50 μM, about 100 nM to about 6.25 μM, or about 200 nM to about 6.25 μM, for example, about 50 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 1 μM, 1.25 μM, 1.5 μM, 1.75 μM, 2 μM, 2.25 μM, 2.5 μM, 2.75 μM, 3 μM, 3.25 μM, 3.5 μM, 3.75 μM, 4 μM, 4.25 μM, 4.5 μM, 4.75 μM, 5 μM, 5.25 μM, 5.5 μM, 5.75 μM, 6 μM, 6.25 μM, 6.5 μM, 7 μM, 7.5 μM, 8 μM, 8.5 μM, 9 μM, 9.5 μM, 10 μM, 10.5 μM, 11 μM, 11.5 μM, 12 μM, 12.5 μM, 13 μM, 13.5 μM, 14 μM, 14.5 μM, 15 μM, 15.5 μM, 16 μM, 16.5 μM, 17 μM, 17.5 μM, 18 μM, 18.5 μM, 19 μM, 19.5 μM, 20 μM, 21 μM, 22 μM, 23 μM, 24 μM, 25 μM, 26 μM, 27 μM, 28 μM, 29 μM, 30 μM, 31 μM, 32 μM, 33 μM, 34 μM, 35 μM, 36 μM, 37 μM, 38 μM, 39 μM, 40 μM, 45 μM, 45 μM, 50 μM, 55 μM, 60 μM, 65 μM, 70 μM, 75 μM or 80 μM. In yet one more example, a medium according to one or more embodiments of the present invention can contain polyamines so as result in a concentration of spermine in the medium of about 0.5 nM to 1 mM, for example, about 0.5 nM, 20.5 nM, 40.5 nM, 60.5 nM, 80.5 nM, 100.5 nM, 120.5 nM, 140.5 nM, 160.5 nM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 μM, 20.5 μM, 40.5 μM, 60.5 μM, 80.5 μM, 100.5 μM, 120.5 μM, 140.5 μM, 160.5 μM, 180.5 μM, 200.5 μM, 220.5 μM, 240.5 mM, 260.5 μM, 280.5 mM, 300.5 μM, 320.5 μM, 340.5 μM, 360.5 μM, 380.5 μM, 400.5 μM, 420.5 mM, 440.5 μM, 460.5 μM, 480.5 μM, 500.5 μM, 520.5 μM, 540.5 mM, 560.5 mM, 580.5 μM, 600.5 μM, 620.5 μM, 640.5 μM, 660.5 μM, 680.5 μM, 700.5 μM, 720.5 μM, 740.5 μM, 760.5 μM, 780.5 μM, 800.5 μM, 820.5 μM, 840.5 mM, 860.5 μM, 880.5 mM, 900.5 μM, 920.5 μM, 940.5 μM, 960.5 μM, 980.5 mM or 1 mM. In yet one more example, a medium according to one or more embodiments of the present invention can contain polyamines so as to result in a concentration of spermidine in the medium of about 0.5 nM to 1 mM, for example, approximately 0.5 nM, 20.5 nM, 40.5 nM, 60.5 nM, 80.5 nM, 100.5 nM, 120.5 nM, 140.5 nM, 160.5 nM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 μM, 20.5 μM, 40.5 μM, 60.5 μM, 80.5 μM, 100.5 μM, 120.5 mM, 140.5 μM, 160.5 mM, 180.5 mM, 200.5 μM, 220.5 mM, 240.5 μM, 260.5 μM, 280.5 μM, 300.5 μM, 320.5 μM, 340.5 μM, 360.5 μM, 380.5 μM, 400.5 μM, 420.5 mM, 440.5 μM, 460.5 μM, 480.5 μM, 500.5 μM, 520.5 mM, 540.5 μM, 560.5 μM, 580.5 μM, 600.5 mM, 620.5 μM, 640.5 mM, 660.5 mM, 680.5 μM, 700.5 μM, 720.5 mM, 740.5 μM, 760.5 μM, 780.5 mM, 800.5 mM, 820.5 μM, 840.5 mM, 860.5 mM, 880.5 μM, 900.5 μM, 920.5 mM, 940.5 μM, 960.5 μM, 980.5 mM or 1 mM. In yet one more example, a medium according to one or more embodiments of the present invention can contain polyamines so as to result in a, concentration of putrescine in the medium of about 0.5 nM to 1 mM, for example, approximately 0.5 nM, 20.5 nM, 40.5 nM, 60.5 nM, 80.5 nM, 100.5 nM, 120.5 nM, 140.5 nM, 160.5 nM, 180.5 nM, 200.5 nM, 220.5 nM, 240.5 nM, 260.5 nM, 280.5 nM, 300.5 nM, 320.5 nM, 340.5 nM, 360.5 nM, 380.5 nM, 400.5 nM, 420.5 nM, 440.5 nM, 460.5 nM, 480.5 nM, 0.5 mM, 20.5 mM, 40.5 μM, 60.5 μM, 80.5 μM, 100.5 μM, 120.5 μM, 140.5 μM, 160.5 μM, 180.5 μM, 200.5 mM, 220.5 mM, 240.5 mM, 260.5 μM, 280.5 mM, 300.5 μM, 320.5 μM, 340.5 μM, 360.5 μM, 380.5 μM, 400.5 μM, 420.5 μM, 440.5 μM, 460.5 μM, 480.5 mM, 500.5 μM, 520.5 μM, 540.5 mM, 560.5 mM, 580.5 mM, 600.5 μM, 620.5 μM, 640.5 μM, 660.5 μM, 680.5 μM, 700.5 mM, 720.5 μM, 740.5 μM, 760.5 μM, 780.5 mM, 800.5 mM, 820.5 μM, 840.5 μM, 860.5 μM, 880.5 μM, 900.5 μM, 920.5 μM, 940.5 μM, 960.5 mM, 980.5 μM or 1 mM.

It is to be understood that media according to the embodiments of the present invention can be provided in a powdered form to be prepared prior to use, in a concentrated form to be diluted prior to use, or in a form to be used without further dilution. The effective amounts discussed above are in reference to the media in its prepared, “working” form, to be used without further dilution. For example, a medium according to the embodiments of the present invention can be a sterile liquid, supplied as a “working solution” to be used without further dilution, in which case the medium contains effective amounts of one or more active agents discussed above. In another non-limiting example, the medium can be a gel containing effective amounts of the one or more active agents. The medium according to the embodiments of the present invention can be a liquid (including a true solution, a suspension and an emulsion), a semi-solid or a solid, such as a gel. When a medium according to the embodiments of the present invention is provided in a form requiring further preparation, such as a powder or a concentrate, one or more active agents are included in amounts or concentration intended to provide a suitable effective amount or amounts after the medium is prepared. For example, a 2× concentrated medium may contain twice the effective amount or amounts of one or more active agents intended to be included in the final “working” form of the medium.

In addition to effective amount or amounts of the one or more active agents discussed above, media according to the embodiments of the present invention contains the components configured to support the at least one mammalian cell in vitro or ex viva. Variations of media are envisioned that can support various types of mammalian cells, such as an embryonic cell, a non-embryonic stem cell, a pluripotent stem cell, an induced pluripotent stem cell, a multipotent stem cell, an adult stem cell, a progenitor cell, a differentiated cell, an isolated primary cell, a secondary cell, an immortalized cell, a cell line cell, a germline cell, a somatic cell, a modified cell. Variations of media are envisioned that can support a single cell, multiple cells, a cell culture, a cell aggregate, a tissue cultures, a tissue, an organ, a blastoderm, an embryoid body or an embryo, including human and non-human embryos.

Media, such as culture media, according to the embodiments of the present invention comprises one or more appropriate nutrient sources for growth and/or maintenance of mammalian cells and maintains appropriate pH and osmolarity. Media can contain natural ingredients, artificial ingredients and/or synthetic ingredients. Examples of natural ingredients are biological fluids (such as plasma, serum, lymph or amniotic fluid), tissue extracts (such as extracts of liver, spleen, tumors, leucocytes, bone marrow or animal embryos). Example of culture media composed of artificial ingredients (“artificial media”) are MEM and DMEM. Artificial culture media can be serum-containing culture media, serum-free culture media (which can contain defined qualities of purified growth factors, lipoproteins and other components provided by the serum), chemically defined culture media or protein-free culture media. Media, such as culture media can comprise one or more of a buffer, one or more inorganic salt, essential amino acids, one or more carbohydrate, such as glucose, fatty acids, lipids, vitamins and trace elements. One example of a buffer is a so-called natural buffering system, in which gaseous CO₂ balances with the CO₃²⁻/HCO₃⁻ content of the culture. Another example is a chemical buffering system, such as the one using 4-(2-hydroxyethyl)-1-piperazineethanesulfonic acid (HEPES), a zwitterionic buffering agent. Media can contain a pH indicator, such as phenol red, which allows pH monitoring during cell growth. Inorganic salt or salts in the media supply sodium, potassium and calcium ions, provide osmotic balance and help regulating cell membrane potential. Essential amino acids, which cannot be synthesized by the cells, are included in the media, but nonessential amino acids may also be included to improve cell growth and viability. Carbohydrates, such as glucose, galactose, maltose or fructose are included as a source of energy. Proteins and peptides, such as albumin, transferrin or fibronectin may also be included, as well as fatty acids and lipids, particularly in serum-free media. Vitamins essential for growth and proliferation of cells, such as B group vitamins, can also be included. Examples of trace elements added to media, particularly serum free media, are copper, zinc, and selenium. Some examples of the media embodiments are based on commercially available media, such as, but not limited to, Essential 8 Medium, CTS Essential 8 Medium, Essential 6 Medium, StemFlex Medium, CTS KnockOut SR Xeno-free Medium, KnockOut Serum Replacement, StemPro, mTeSR, mTeSR1, StemFit, Nutristem, Neurobasal or BrainPhys, comprising effective amount or amounts of the one or more active agents described in this document.

Compositions according to the embodiments of the present invention include in vitro or ex viva compositions comprising a medium according to an embodiment of a present invention and at least one mammalian cell. At least one mammalian cell in such embodiments can be one mammalian cell or a plurality of mammalian cells of the same or different type. For example, a mammalian cell can be an embryonic cell, a non-embryonic stem cell, a pluripotent stem cell, an induced pluripotent stem cell, a multipotent stem cell, an adult stem cell, a progenitor cell, a differentiated cell, an isolated primary cell, a secondary cell, an immortalized cell, a cell line cell, a germline cell, a somatic cell or a modified cell. A plurality of mammalian cells can be multiple cells, a cell culture, a cell aggregate, a tissue culture, a tissue, an organ, a blastoderm, an embryoid body or an embryo, including human and non-human embryos. At least one mammalian cell can be thawed. It is understood that some of the embodiments of the present invention that comprise a medium and at least one mammalian cell can further comprise a vessel containing the medium, such as a bag, a flask, such as culture flask, a dish, such as a culture dish, a tube or a reactor. It is understood that some of the embodiments of the present invention that comprise a medium and at least one mammalian cell can further comprise a support or a scaffold for cells. Some non-limiting examples of the embodiments of the present invention comprising a culture medium and at least one mammalian cell are E8 Essential medium and at least one pluripotent stem cell, mTeSR medium and at least one pluripotent stem cell, StemPro and at least one pluripotent stem cell, E6 Essential medium and at least one embryoid body, Neurobasal and at least one neuron, BrainPhys and at least one neuron.

### Kits

Kits for cell, tissue or organ culture, maintenance and/or preservation are included among embodiments of the present invention. A kit is a set of components, comprising at least some components for culturing, maintaining or preserving cells (including single cells and groups of cells), tissues or organs. Such a kit contains one or more active agents discussed in the “Small molecules” section of this document. A kit may contain further one or more of the following: a media configured to support at least one mammalian cell in vitro or ex vivo or one or more of culture media components; a vessel for holding the medium; a culture vessel, such as a flask, a dish, a plate (including a multi-well plater) or a reactor; a support or a scaffold for cell, tissue or organ culture. A kit may contain one or more mammalian cells. Mammalian cell or cells included in a kit can be one or more of an embryonic cell, a non-embryonic stem cell, a pluripotent stem cell, an induced pluripotent stem cell, a multipotent stem cell, an adult stem cell, a progenitor cell, a differentiated cell, an isolated primary cell, a secondary cell, an immortalized cell, a cell line cell, a germline cell, a somatic cell or a modified cell. One or more mammalian cells can be provided in a frozen or non-frozen form. Examples of other components that can be included a kit are modulators of biological signaling pathways, including small molecules (for example, CHIR99021 for WNT pathway activation) or recombinant proteins (for example, WNT3A, Sonic Hedgehog, Bone Morphogenetic Proteins or Activin A). Some kits may contain one or more compositions described in the “Compositions” section of this document.

For example, a kit may contain a medium described in the “Compositions” section of this document, a vessel for cell culture and optionally one or more mammalian cells suitable for being cultured in the medium included in the kit. A kit may contain a culture medium described in the “Compositions” section of this document, a vessel for cell culture and at least one of a support of a scaffold, and optionally one or more animal cells suitable for being cultured in the culture medium included in the kit. One example is a kit comprising Essential 8 Medium, one or more active agents according to the embodiments of the present invention (which can be included in the culture medium or supplied as a separate kit component to be added to the culture medium prior to use, and cell culture plates or flasks of various size coated with vitronectin, laminin 521 or Matrigel. In one more example, a kit may contain a medium described in the “Compositions” section of this document and a vessel for maintenance or preservation in the medium of the cells, groups of cells, tissues, embryos, organs or organ parts.

### Uses and Methods

Various uses and methods (processes) of using of the compositions and kits discussed throughout this document are envisioned and included among the embodiments of the present invention. For example, various combinations of the compounds described in the “Small Molecules” section of this document, which can be referred to as “active agent” or “active agents,” can be advantageously used in methods of culturing, maintaining or preserving animal cells, including, but not limited to, stem cells (for example, embryonic stem cells, non-embryonic stem cell, pluripotent stem cells, induced pluripotent stem cells, a multipotent stem cells and adult stem cells) progenitor cells, differentiated cells, isolated primary cells, secondary cells, immortalized cells, cell line cells, germline cells, somatic cells and various types of modified cells (including iFSCs and genetically modified or engineered cells). Methods of culturing, maintaining or preserving mammalian cells according to the embodiments of the present invention include, but are not limited to, the methods that involve one or more of growth of cells, proliferation of cells, differentiation of cells, dedifferentiation of cells, induction of potency (including pluripotency) in cells, single cell cloning (culture), crypreservation, and maintenance of cells in culture. Ex vivo preservation of cells, groups of cells, tissues, organ parts, organs or embryos is included among the methods according to the embodiments of the present invention. Methods of culturing mammalian cells according to the embodiments of the present invention can use a single cell or a plurality of cells as one or more of a starting material, a process intermediate or an end product. A plurality of mammalian cells can be multiple cells (for example, a suspension of dissociated cells or a suspension cell culture), a cell culture (such as an adherent or non-adherent cell culture), a cell aggregate, a tissue culture, a tissue (including an artificially engineered cultured tissue), an organ, a blastoderm, an embryoid body or an embryo, including human and non-human embryos. Some of the above-listed categories of cells and pluralities of cells may overlap. Embodiments of the method of the present invention can lead to improved outcomes, measured by appropriate end-points (for example, cell survival, cell differentiation or dedifferentiation, cell proliferation etc.) of culturing of both non-stem and stem cells (such as ESCs, iPSCs, adult stem cells including neural stem cells, hematopoietic stem cells, mesenchymal stem cells and other organ-derived stem cells or artificially modified stem cells).

Various uses and applications of the methods according to the embodiments of the present invention are envisioned. In one example, the methods according to the embodiments of the present invention can be incorporated into cell reprogramming processes, including processes for generating iPSCs and establishing iPSC clones. In another example, the methods according to the embodiments of the present invention can be incorporated in the processes of establishing new cell lines, including the cell lines derived from primary cells, including cancer cells and no-cancerous cells, and hybridoma cell lines. Mammalian cells cultured according to the methods of the present invention can be thawed prior to culturing or frozen upon culturing. Accordingly, some embodiments of methods of the present invention can include a step of thawing, a step of freezing or both. Thus, some embodiments of the methods of the present invention can be incorporated into processes of cryopreservation and/or thawing of cells. In one more example, embodiments of the methods of the present invention can be incorporated into processes or growth, proliferation and/or differentiation of cells in vitro and ex vivo in two- and three-dimensional cultures, including the processes used for tissue, organoid and organ culturing, growth and/or engineering. In yet another example, embodiments of the methods of the present invention can be incorporated into processes or preservation and/or maintenance of cells or groups of cells in vitro and/or ex vivo including the processes used for in vitro and/or ex vivo for maintenance and preservation of cells, tissues, organoids, embryos, organ parts or organoids. In some examples, in vitro and/or ex vivo maintenance or preservation methods are used prior to transplantation of of cells, tissues, organoids, embryos, organ parts or organoids into a host. In one more example, embodiments of the methods of the present invention can be incorporated into processes for genetic modification, such as genome editing of mammalian cells. In one more example, embodiments of the methods of the present invention can be incorporated into processes for clone selection of mammalian cells, which includes the processes and procedures referred to as single-cell cloning. The methods according to the embodiments of the present invention can be advantageously used to improve the outcomes of single-cell cloning in a variety of processes, such as genome editing of iPSCs for preclinical research and clinical applications. Some examples of such applications are correcting a genetic defect through personalized cell therapy, introducing a genetic mutation for disease modeling or introducing a transgene to generate a reporter cell line for drug discovery. In one more example, embodiments of the methods of the present invention can be incorporated into processes for producing embryoid bodies from mammalian cells.

Methods according to the embodiments of the present invention can be used, among other things, for efficient generation of new iPSC lines from somatic cells (such as skin fibroblasts or blood cells), growing, expanding and/or differentiating iPSCs or embryonic stem cells; efficient utilization of different cell types differentiated from iPSCs and/or embryonic stem cells (such as neurons, astrocytes, oligodendrocytes, retinal pigment epithelium, hepatocytes, cardiomyocytes or pancreatic beta cells capable of producing insulin) and/or establishing new tumor cell lines from patient material. Methods according to the embodiments of the present invention can use a wide array of media typically used in mammalian cell culture.

Methods of producing, obtaining or preparing a culture medium incorporating various combinations of the compounds described in the “Small Molecules” section of this document, which can be referred to as “active agent” or “active agents,” are also included among the methods according to the embodiments of the present invention. Such methods may include a step or steps of combining one or more components of the culture medium with one or more active agents according to the embodiments of the present invention.

### Systems

Systems for performing the methods of the present invention are included among the embodiments of the present invention. These systems include various stations and/or components. As used herein, the term “station” is broadly defined and includes any suitable apparatus or assemblies, conglomerations or collections of apparatuses or components suitable for carrying out a method according to the embodiments of the present invention. The stations need not be integrally connected or situated with respect to each other in any particular way. Systems according to the embodiments of the present invention may include any suitable arrangements of the stations with respect to each other. For example, the stations need not even be in the same room. But in some embodiments, the stations are connected to each other in an integral unit.

For example, a system embodiment may include a station for robotic or automated cell culture such as the CompacT SelecT™ (Sartorius, Wilmington, Del.) that can grow, expand, and differentiate iPSCs or embryonic stem cells or cancer cell lines. A system embodiment may include a station for generating reports. A system embodiment may include a station or components for data analysis. A system embodiment may include a computer, a processor, electronic memory, software instructions etc. A system embodiment, or parts of the system embodiment, may be controlled by a computer.

### Computer-Based Calculations and Tools

The methods described in this document can involve computer-based calculations and tools. Tools can be advantageously provided in the form of computer programs that are executable by a general-purpose computer system (which can be called “host computer”) of conventional design. The host computer may be configured with many different hardware components and can be made in many dimensions and styles (for example, desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the present invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (for example, using PERL), execution of which can be initiated by a user from a standard command line interface of an operating system such as Linux or UNIX. Commands can be adapted to the operating system as appropriate. In other embodiments, a graphical user interface may be provided, allowing the user to control operations using a pointing device. Thus, the present invention is not limited to any particular user interface.

Scripts or programs incorporating various features of the present invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### Advantages

The compositions, kits and methods described herein offer a number of significant improvements over the previously known compositions, kits and methods. One of the advantages is unexpected potency and specificity of Chroman 1, when used as a ROCK kinase inhibitor. Another advantage is unexpected synergism exhibited by Chroman 1 and caspase inhibitor Emricasan or its derivative Q-VD-OPh for improving cell culture outcomes, including survival of cells during routine cell culture, cell expansion maintaining normal karyotype, culturing of thawed cryopreserved cells, and cell differentiation in monolayer cultures, embryoid bodies, neurospheres or organoids. One more advantage are unexpected superior effects of the combination of Chroman 1, Emricasan, trans-ISRIB and polyamines on cell culture outcomes, particularly during cell sorting, single-cell cloning, cell reprogramming, cryopreservation and cell thawing. The above unexpected advantages of the active agents according to the embodiments of the present invention allow them to be beneficially employed in various processes and methods using cell, tissue and organ culture. For example, the dosing of the culture can be performed at low concentrations of the one or more active agents, thus leading, among other things, to significant financial savings. In another example, the compositions, methods and kits described herein achieve significantly improved outcomes of long-term cell line expansion in culture and minimize off-target effects of cell culture additives on the cellular machinery of the cultured cells. Profiling the inhibitory activity of Chroman 1 against a set of human protein kinases demonstrated the compound's superior properties as a culture medium additive, in comparison to currently used additives, such as Y-27632, thiazovivin, pyrintegrin and Revitacell® (Thermo Fisher Scientific, Frederick, Md.) and CloneR® (StemCell Technologies, Vancouver, Canada). The compositions, kits and methods described herein also achieve highly improved outcomes of single cell cloning.

The following examples will serve to further illustrate the present invention without, at the same time, however, constituting any limitation thereof. On the contrary, it is to be clearly understood that resort may be had to various embodiments, modifications and equivalents thereof which, after reading the description herein, may suggest themselves to those skilled in the art without departing from the spirit of the invention.

### Example 1

### Quantitative High-Throughput Screening for Compounds Promoting Human Pluripotent Stem Cell Survival after Single-Cell Dissociation

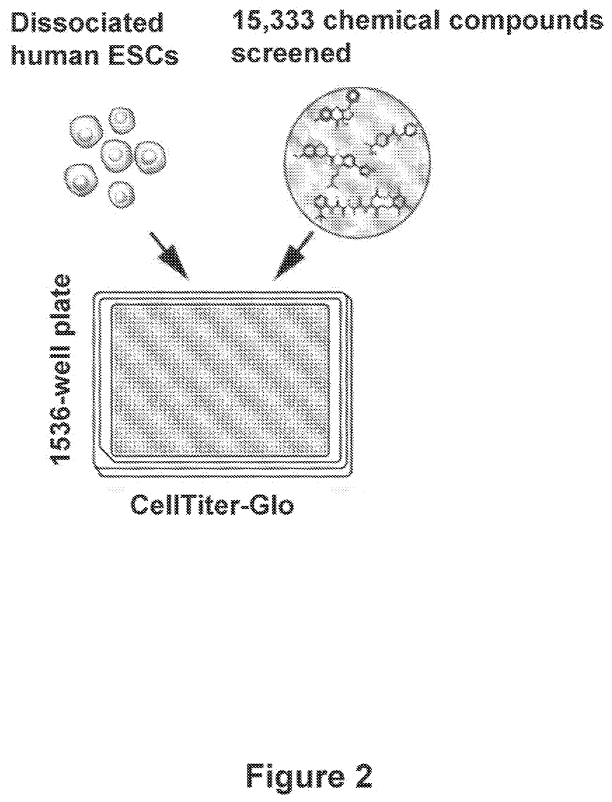

Quantitative high-throughput screening (qHTS) was performed, which identified the compounds promoting embryonic stem cell survival after single-cell dissociation. FIG. 2 schematically illustrates qHTS procedure. H9 cells (official designation WA09, WiCell, Wisconsin) were enzymatically dissociated with Accutase (Thermo Fisher Scientific) and dispensed into 1536-well plates coated with recombinant vitronectin and containing 15,333 small molecule compounds at various concentrations. The small molecule compounds were sourced from small molecule libraries, which included the NCATS Pharmacologically Active Chemical Toolbox (NPACT), the NCATS Pharmaceutical Collection (NPC), the Mechanism Interrogation PlatE (MIPE), as well as commercially available libraries Tocriscreen® Plus from Tocris (Minneapolis, Minn.) and LOPAC1280® from Sigma-Aldrich (St. Louis, Mich.). Dimethyl sulfoxide (DMSO; final concentration 0.4%) and 10 μM Y-27632 were used as the negative and positive controls, respectively, in the screen. 24 hours after seeding, cell viability was assessed with CellTiter-Glo® luminescent cell viability assay (Promega Corporation, Madison, Wis.), which measures cellular adenosine triphosphate (ATP) level as a surrogate for viable cells. qHTS procedure is generally described in Inglese el al. (2006) “Quantitative high-throughput screening: A titration-based approach that efficiently identifies biological activities in large chemical libraries” Proc. Natl. Acad. Sci. USA 103(31):11473-11478 (2006).

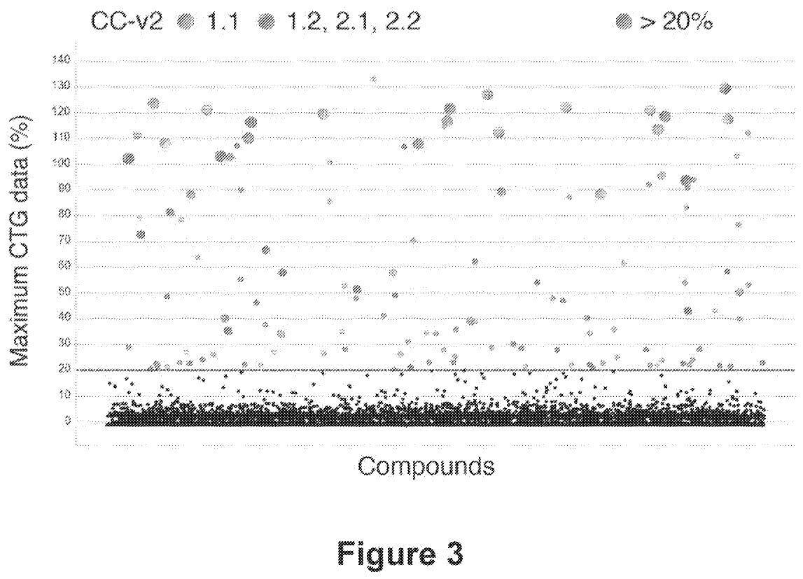

FIG. 3 is a scatter plot of maximum survival achieved by the screened compounds (plotted on the X-axis). All the screed compounds were received pre-plated at the pre-determined concentrations. To generate the scatter plot of FIG. 3, the survival values were normalized based on the survival achieved at 10 μM Y-27632 (taken to be 100%) and plotted at the Y-axis. Different types of dots indicated in the plot represent the compounds with dosage curve classes 1.1, 1.2, 2.1 and 2.2, as indicated. The dots labeled “>20%” represent the compounds that led to maximal survival above the 20% cutoff but did not fall in curve classes 1.1, 1.2, 2.1 or 2.2. A threshold set at 20% survival identified 128 active compounds listed in Table 1. Curve classification is explained in Huang et al. “Chemical genomics profiling of environmental chemical modulation of human nuclear receptors” Environ. Health. Perspect. 119(8): 1142-1148 (2011), in particular in section “qHTS Data Analysis” and Table 3 of “Supplementary Materials.”

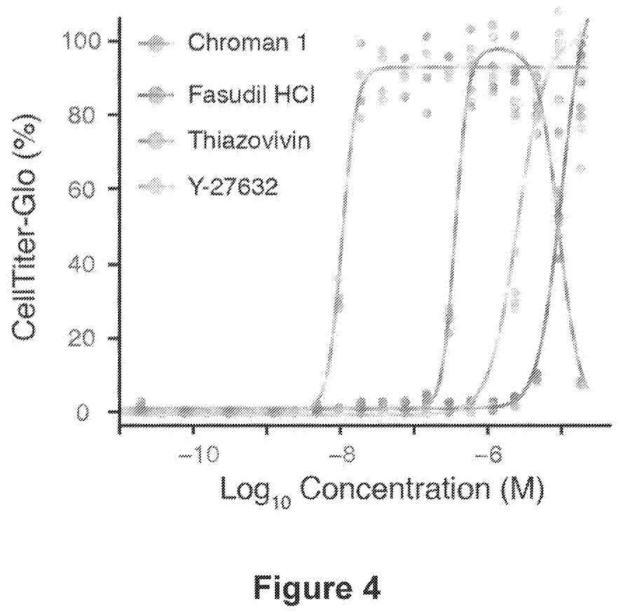

FIG. 4 shows line plots of the dose-response curves of selected active compounds known to inhibit ROCK, such as Chroman 1, Fasudil HCL, Thiazovivin and Y-27632. The direct comparison illustrated in FIG. 4 showed the superiority of Chroman 1 compared to the other three ROCK inhibitors previously used in the stem cell field. Molar concentrations of the selected ROCK inhibitors are plotted on the X-axis (log scale). Four replicates were tested for each concentration, and the data were normalized with respect to the average CellTiter Glo™ (CTG) reading obtained from 10 μM Y-27632. Thusly normalized data were plotted on the Y-axis. The dose-response experiments clearly demonstrated that Chroman 1 was more potent than to Y-27632, Thiazovivin or Fasudil.

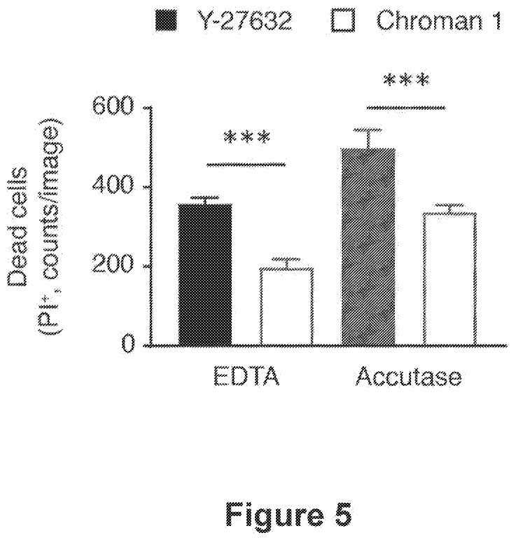

FIG. 5 illustrates the direct comparison of the effects of 10 μM Y-27632 and 50 nM Chroman 1 on cell survival (higher number of propidium iodide-positive dead cells). H9 cells were dissociated with 0.5 mM ethylenediaminetetraacetic acid (EDTA) or Accutase (enzymatic dissociation) according to standard procedures and seeded into vitronectin coated 6-well plates. Phase-contrast and fluorescence microscopy digital images were taken 12 hours after cell plating (data not shown). To generate the fluorescence microscopy images, live cells were stained with Calcein (0.5 μg/ML; Thermo Fisher Scientific, catalog number: C34852), and dead cells were stained with propidium iodide (PI; 500 nM; Thermo Fisher Scientific, catalog number: P3566). For quantitative analysis, the cells were counted using an automated cell counter (Cellometer Auto 2000, Nexcelom). The results of PI staining are illustrated in the bar graph shown in the right panel of FIG. 5. The above experiments showed that Y-27632 treatment led to higher number of dead cells than Chroman 1 treatment.

### Example 2

### Chroman 1 is a More Potent and More Specific ROCK Inhibitor than Y-27632

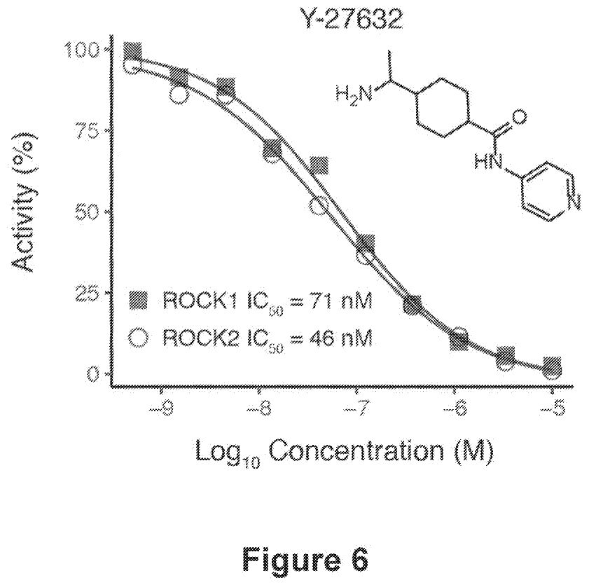

Experimental studies were performed, which demonstrated that Chroman 1 is a more potent and more specific ROCK inhibitor than Y-27632. The half maximal inhibitory concentrations (IC₅₀) of each of Y-27632 and Chroman 1 were determined in kinase assays against their primary targets ROCK1 and ROCK2 using the HotSpot Kinase Assay performed by Reaction Biology Corporation (Malvern, Pa.). The results are illustrated by the line plots shown in FIGS. 6 (Y-27632) and 7 (Chroman 1). Chroman 1 was determined to be a more potent ROCK inhibitor than Y-27632.

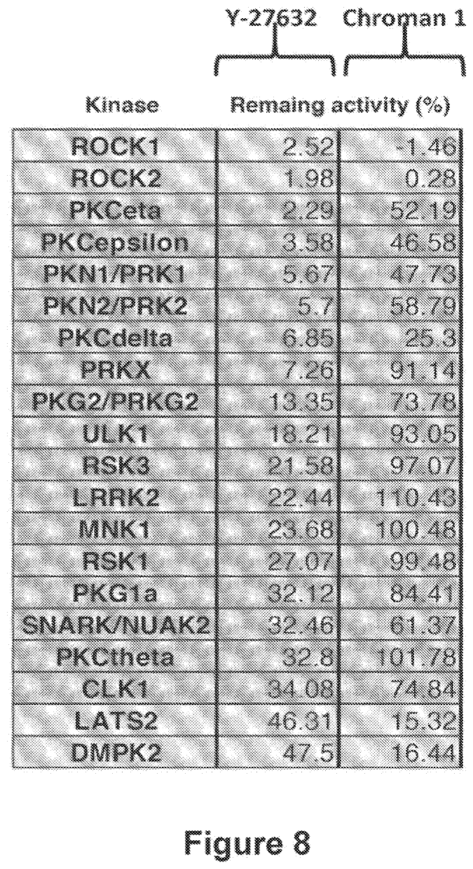

The results of kinase profiling experiments are illustrated in FIG. 8. Y-27632 and Chroman 1 kinase profiling was performed by testing their inhibitory activity on the human kinases using HotSpot Kinase Assay provided by Reaction Biology Corporation. Y-27632 was tested at 10 μM and Chroman 1 at 50 nM. The phylogenic trees of human kinases were generated based on a neighbor-joining tree via ClustalW software derived from multiple protein sequence alignment of human kinase domains (Manning et al., “The protein kinase complement of the human genome” Science 298:1912-1934 (2002)). Kinase profiling revealed higher specificity of Chroman 1 based on less off-target inhibition across all human kinases tested. The values under 10% of original activity were deemed to represent significant inhibitor activity (based on Anastassiadis et al. “Comprehensive assay of kinase catalytic activity reveals features of kinase inhibitor selectivity.” Nat. Biotechnol. 29, 1039-1045 (2011).). ROCK1 and ROCK2 were identified the primary targets for both Y-27632 and Chroman 1. Y-27632 was shown to have a number of off-targets, including PKCeta (also known as PKC η or PRKCH), PKCepsilon (also known as PKC ε or PRKCE), PKCdelta (also known as PKC δ or PRKCD), PKN1, PKN2 and PRKX.

### Example 3

### Combinatorial Matrix Screening and Improved Cell Survival with Chroman 1 and Emricasan

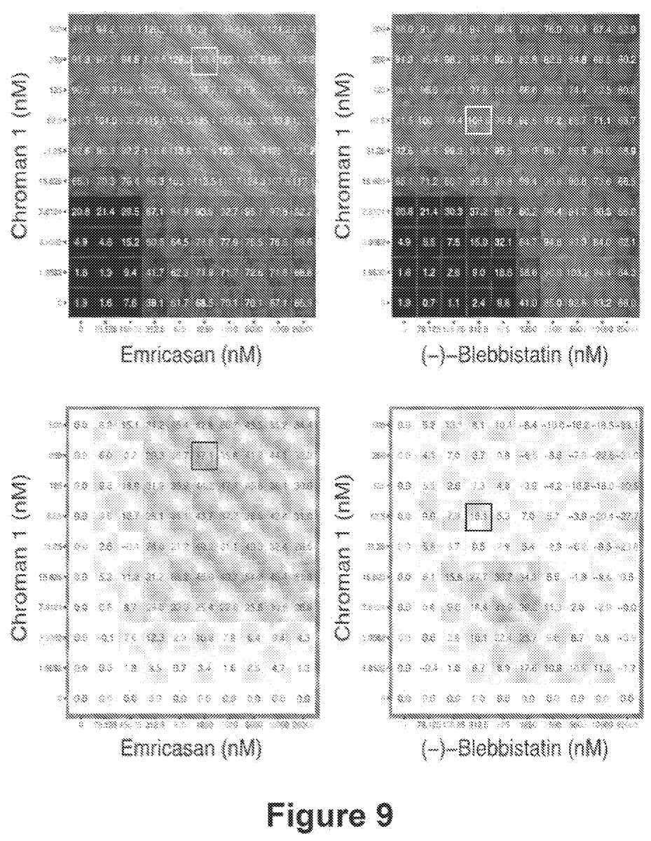

Combinatorial matrix screening was performed to identify the compounds with synergistic effects on cell survival. Focusing on the compounds with different mechanisms of action, 29 compounds were selected for combinational matrix screening, resulting in 812 sets of 10×10 checkerboard matrix experiments (data not shown), four of which are illustrated in FIG. 9. The concentrations of the compounds for combinational matrix screening were optimized based on the slope and potency (AC₅₀) derived from single-agent qHTS dose-response curves, so that the matrix screen covered a wide range of biological effect observed in the cell viability assay. The cell survival achieved by 10 μM Y-27632, used alone, was set as the reference for 100% cell survival and used to normalize all the cell survival values. Caspase inhibitor Emricasan and its derivative Q-VD-OPh were identified, by combinatorial matrix screening, as synergistic with Chroman 1. FIG. 9 shows the results of combinatorial matrix screening for the combination of Chroman 1 and caspase inhibitor Emricasan and the combination of Chroman 1 and (−)-Blebbistatin. Blebbistatin is an inhibitor of non-muscle myosin II ATPases and was included to demonstrate the difference between the compound pairs that synergize (Chroman 1 and Emricasan) and compound pairs lacking synergy (Chroman 1 and Blebbistatin). The top matrices shown in FIG. 9 show the actual cell survival data normalized to the survival achieved at 10 μM Y-27632 (taken at 100%). The numerals in each 10×10 matrix show the maximum survival achieved with the indicated combinations or single agents. The maximum survival achieved by each pair of compounds is represented by the highest numbers in each 10×10 matrix, the examples of which are shown in FIG. 9. The bottom matrices depict the synergy matrix calculated using highest-single agent (HSA) model. The HSA synergy assesses the pro-survival benefit of compound combination compared to single agent alone, which was computed as the difference between cell survival out of combination and that from the best single agent at each tested concentration. Synergy is defined as the difference between the combination and max response with single agent (synergy=combination max (single agent A, single agent B).

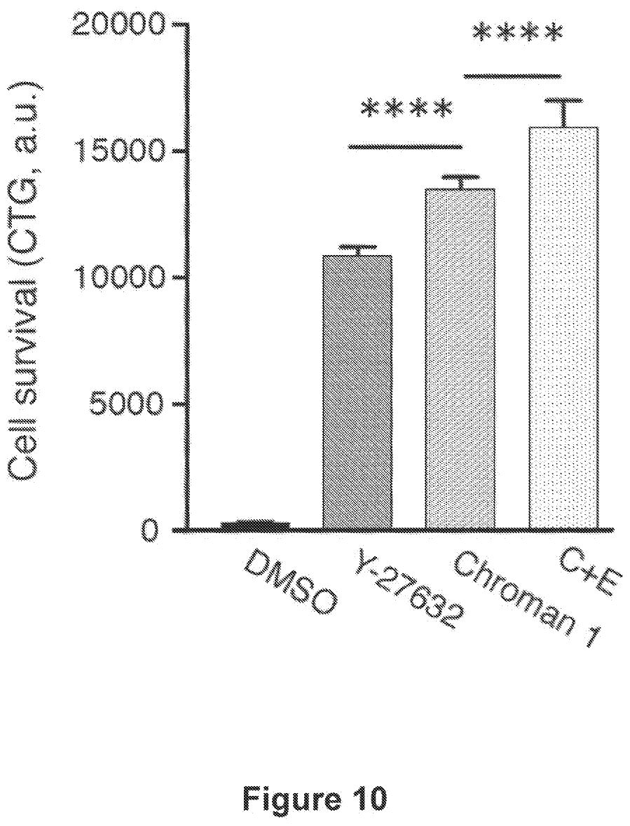

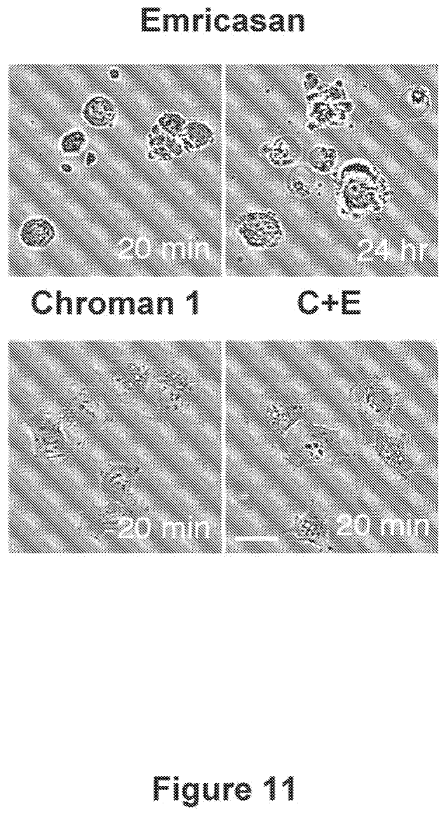

FIG. 10 illustrates improved cell survival when Chroman 1 and Emricasan were combined. CellTiter-Glo® assay was used to quantify viable 119 cells 24 hours post-plating (100,000 cells/cm⁶). FIG. 11 shows phase-contrast microscopy images of a time-lapse experiments monitoring cell behavior over 24 hours (IncuCyte Zoom® Live Cell Analysis, Sartorius, DE). To obtain the images, single-cell dissociated 119 cells were seeded on vitronectin-coated plates in the presence of the compounds indicated (C+E stands for a combination of 50 nM Chroman 1 and 5 μM Emricasan) and monitored over time. Cellular stress was already apparent 20 minutes after plating in the presence of Emricasan, which ultimately led to dead cells at 24 hours. In contrast, when using Chroman 1 (inhibitor of cell contractility) alone or in combination with Emricasan shows cell attachment already 20 min after plating. The above experiments demonstrated that the combination of Chroman 1 and Emricasan was essential for maximum efficiency of cell attachment and survival. The experimental results illustrated in FIG. 11 showed that Emricasan, although capable of blocking caspase 3 activation, did not prevent cell contraction, ultimately resulting in cell death. Thus, the presence of a caspase inhibitor Emricasan alone was not sufficient to improve cell viability beyond 24 hours. Without ROCK kinase inhibition using Chroman 1, cells continued to contract, did not attach to the coating substrate and ultimately underwent cell death.

### Example 4

### Improved Cell Survival after Routine Passage and Thawing of Cryopreserved Cells Treated with Chroman 1 and Emricasan Combination

Experimental studies discussed below showed improved cell survival of the cells treated with Chroman 1 and Emricasan combination. In one series of experiments, 1-19 cells were single-cell dissociated and seeded onto vitronectin-coated plates at 100,000 cells per cm². Caspase 3/7 green detection reagent was used to continuously monitor caspase activation and apoptosis over time by means of microscopic imaging (IncuCyte Zoom®—data not shown. The majority of cells underwent apoptosis in the presence of 0.0001% v/v DMSO. Addition of 10 μM Y-27632 was beneficial, while using 50 nM Chroman 1 further improved cell survival. The combined use of 50 nM Chroman 1 and 5 μM Emricasan maximized the cell survival effect. CellTiter-Glo® assay (CTG) was used to measure viable cells 24 hours after seeding (data not shown). The above-discussed experimental study of dissociated H9 cells showed that the combination of Chroman 1 and Emricasan led to superior cell survival, in comparison to a positive control Y-27362 or Chroman 1 used alone.

In another series of experiments, frozen H9 cells were thawed and seeded onto vitronectin-coated plates at 100,000 cells per cm². As in the above-described study of dissociated cells, caspase 3/7 green detection reagent was used to monitor caspase activation and apoptosis over time (data not shown). CTG assay was used to measure viable cells 24 hours after seeding. The study of the thawed cells showed that the combination of 50 nM Chroman 1 and 5 μM Emricasan led to superior cell survival, in comparison to a positive control 10 μM Y-27362 or Chroman 1 used alone.

To demonstrate that Chroman 1 alone or in combination with Emricasan can be safely used in long-term cell culture, long-term passaging of H9 cells (40 passages) was performed. At every passage, the cells were treated for 24 hours with 50 nM Chroman 1 or a combination of 50 nM Chroman 1 and 5 μM Emricasan. Pluripotent cells maintained a normal karyotype. Immunocytochemical analysis using specific antibodies demonstrated that long-term expanded 1-19 cells expressed typical pluripotency-associated genes (OCT4, NANOG) and were able to differentiate into different lineages. To detect the lineage markers, immunocytochemistry was performed using cell type-specific markers and images were generated using a fluorescence microscope (data not shown). The lineage markers used were PAX6 for ectoderm, Brachyury for mesoderm and SOX17 for endoderm.

### Example 5

### Improved Cell Differentiation in the Presence of Chroman 1 and Emricasan

Improved cell differentiation in the presence of Chroman 1 and Emricasan was demonstrated by embryoid body formation assays. In one series of experiments, H9 cells were dissociated with Accutase. After cell-counting to ensure that the same number of cells was used for different conditions tested, the cells were plated into ultra-low attachment 6-well plates in the presence of 0.0001% v/v DMSO (negative control), 10 μM Y-27632 (positive control), 50 nM Chroman 1, 5 μM Emricasan or a combination of 50 nM Chroman 1 and 5 μM Emricasan. Embryoid body (EB) formation was examined 24 hours after cell seeding. Microscopic examination of the plates indicated that the combination of Chroman 1 and Emricasan dramatically improved EB formation and reduced the number of dead cells and debris compared to DMSO, Y-27632, and Chroman 1 (data not shown).

In a different experimental approach (data not shown), a defined number of H9 cells was plated into AggreWell™ plates (StemCell Technologies, Vancouver, Canada) to generate single EBs per well. Embryoid body (EB) formation was examined 24 hours after cell seeding. Dead cells surrounded the EBs treated with 10 μM Y-27632, whereas the amount of dead cells was significantly lower after 50 nM Chroman 1 treatment. Exposure to 5 μM Emricasan alone did not result in proper cell aggregation and EB formation. Importantly, the combination of 50 nM Chroman 1 and 5 μM Emricasan generated a high-quality EB with virtually no dead cells detectable.

Pluripotent stem cells (WA09, also called H9 cells, obtained from WiCell, Madison, Wis.) dissociated with Accutase were seeded into AggreWell™ plates (StemCell Technologies, Vancouver, Canada) to generate EBs. Bright field images were taken 24 hours after seeding. Diameter comparison (data not shown) of EBs under different treatment conditions showed that larger EBs were consistently generated by using 50 nM Chroman 1 and 5 μM Emricasan, in comparison either to either 50 nM Chroman 1 alone or 10 μM Y-27632.

H9 cells dissociated with Accutase were seeded into 96-well plates (ultra-low attachment) to generated EBs. After 24 hours, calcein (green staining, live cells) and PI (red staining, dead cells) were added to the cell culture medium to visualize live and dead cells in the wells. Quantification of the live cells by CTG measurement confirmed that a combination of 50 nM Chroman 1 and 5 μM Emricasan resulted in the highest cell survival of all the conditions tested. CTG measurement (ATP levels) (data not shown).

### Example 6

### Improved Multi-Lineage Differentiation of Pluripotent Cells

Improved multi-lineage differentiation of pluripotent cells in the presence of 50 nM Chroman 1 and 5 μM Emricasan was experimentally demonstrated (data not shown). Pluripotent stem cells (H9 from WiCell) dissociated with Accutase were seeded into 96-well ultra-low attachment plates to generate EBs and cultured in Essential 6 Medium (Thermo-Fisher Scientific, Frederick, Md.) to induce spontaneous differentiation. mRNA expression of ectoderm marker PAX6, mesoderm maker Brachyury and endoderm marker Sox17 were quantified using qPCR for individually grown EBs on post-seeding day seven. Actin mRNA expression was used to normalize cell number variation among EBs. All lineage-specific markers were expressed at higher levels when a combination of 50 nM Chroman 1 and 5 μM Emricasan (Chroman 1 Emri) were used.

The EB formation assay is a widely used approach intended to measure pluripotency by demonstrating differentiation into ectoderm, mesoderm and endoderm. Analysis and quantification of individually grown EBs indicated that using Chroman 1 and Emricasan in combination substantially increased the percentage of EBs that expressed all three markers of multi-lineage differentiation (PAX6, Brachyury, SOX17) (data not shown). This finding indicated that using Chroman 1 and Emricasan increased the robustness of the EB formation assay and reduced an inherent systematic error due to cell death. Hence, combined use of Chroman 1 and Emricasan improves the EB formation assay and helps to standardize the analysis of the true differentiation potential of pluripotent cell lines.

### Example 7

### Identification of Trans-ISRIB and Polyamines as Additional Cell Survival-Promoting Factors

To extend and maximize the use of Chroman 1 and Emricasan for low cell seeding density conditions and single cell cloning, we developed another combinatorial screening strategy. Experimental studies were performed that identified Trans-ISRIB and polyamines as additional cell survival-promoting factors. An assay was developed to screen for compounds that are capable of overcoming cellular stress associated with low cell seeding density. The experiments aimed to define the detection limit and signal strength of the CTG assay in order to detect single cells per well. H9 cells (obtained from WiCell) were used. The experimental results showed that cellular stress associated with low seeding density was apparent when fewer than 50 cells were seeded in each well of a 1536-well plate (data now shown).

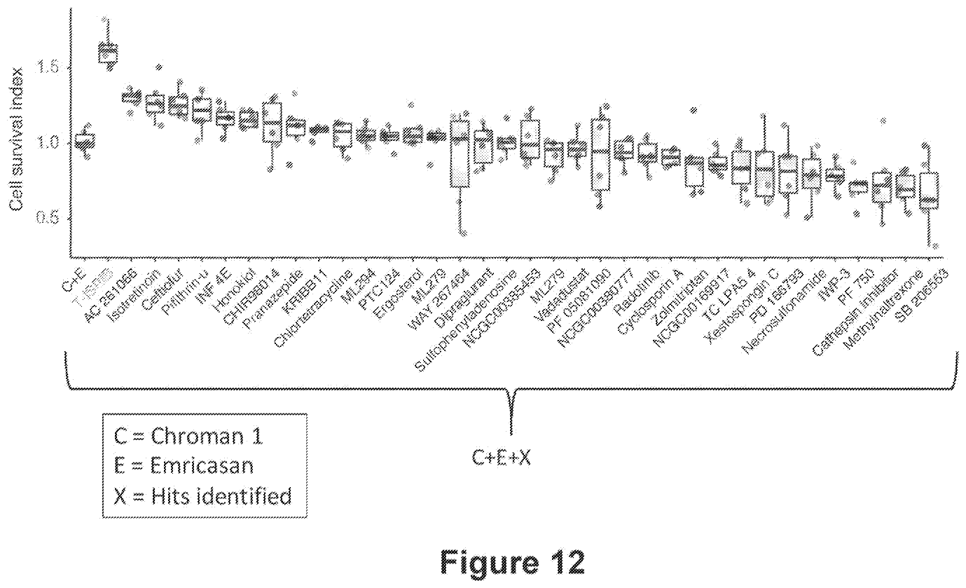

The seeding density of 10 cells-per-well was used to screen additional compounds with potential synergy with Chroman 1 and Emricasan. The compounds and the concentrations at which they were tested are shown in Table 2. The compounds were all tested in the presence of 50 nM Chroman 1 and 5 μM Emricasan. FIG. 12 illustrates the validation of the compounds initially identified by qHTS in 6-well format, which demonstrated the cell survival-promoting effect of Trans-ISRIB combined with 50 nM Chroman 1 and 5 μM Emricasan. Dissociated stem cells were plated onto vitronectin-coated 1536-well plates at the per-well densities indicated on the X-axis. All the plates were prepared in duplicates. One set of plates was read with CTG right after dispensing (r1), and the other set was read 24 hours after (r2). The CTG fold change plotted on Y-axis was calculated as the ratio of r2 to r1 (r2/r1) to reflect cell survival and growth within 24 hours. Y-axis indicates ratio of improved cell survival, measured by cell confluency, compared to Chroman 1 plus Emricasan, which was used as a baseline. X-axis shows the compounds that were combined with Chroman 1 and Emricasan. The middle line within the box is the median cell growth. The upper and lower box hinges represent the 75^{th} and 25^{th} percentiles, whereas the upper and lower whiskers extend from the box hinge to the largest or lowest value no further than 1.5× distance between 25^{th} and 75^{th} percentile.

The experiments were performed validating the “hits” obtained from the primary screening. Pluripotent stem cells (H9 cells obtained from WiCell) were dissociated using TrypLE (Thermo-Fisher) and seeded into 6-well plates at a density of 25 cells/cm′. The combination of 50 nM Chroman 1, 5 μM Emricasan, Polyamines (40 ng/mL putrescine, 4.5 ng/mL spermidine, 8 ng/mL spermine) and 0.7 μM Trans-ISRIB (the combination referred to by the abbreviation “CEPT”) resulted in the highest number of colonies, when compared to the other conditions tested in these experiments (data now shown).

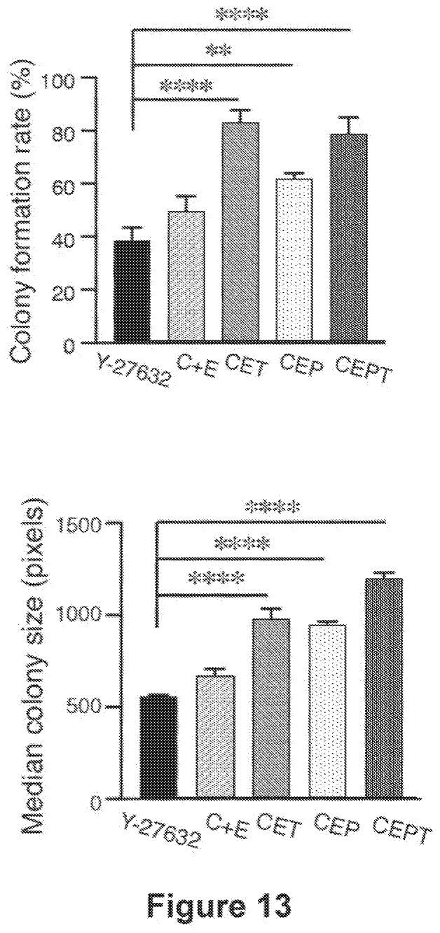

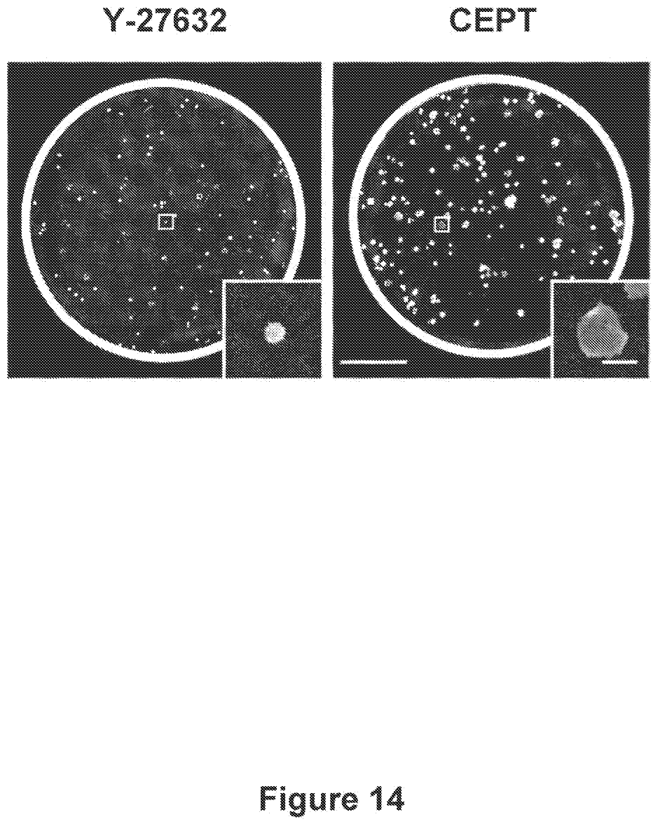

FIG. 13 illustrates colony formation rate and colony size of H9 cells treated with different small molecules and small molecule combinations, denoted as follows (CEPT—50 nM Chroman 1, 5 μM Emricasan, Polyamines (40 ng/mL putrescine, 4.5 ng/mL spermidine, 8 ng/mL spermine), 0.7 μM Trans-ISRIB, C+E—50 nM Chroman 1, 5 μM Emricasan; CET—50 nM Chroman 1, 5 μM Emricasan, 0.7 μM Trans-ISRIB; CEP—50 nM Chroman 1, 5 μM Emricasan, Polyamines (40 ng/mL putrescine, 4.5 ng/mL spermidine, 8 ng/mL spermine, Y-27632—10 μM). FIG. 14 shows representative microscopic images of the colonies that were obtained with Y-27632 and CEPT. Such microscopic images where quantitavely analyzed to produce the data plotted in the bar graphs shown in FIG. 13. Whole-well images (6-well plate) were captured with calcein green (0.5 μg/mL; Thermo Fisher Scientific, catalog number: C34852).

Systematic comparison of small molecule combinations and their effect on colony number and colony size of hESCs plated on vitronectin at 25 cells/cm² in StemFlex medium was performed. The quantification of the colony formation rate and median colony size was performed 6 days after plating. CET, CEP, and CEPT combinations were shown to have superior effects on both colony formation rate and median colony size, when compared to Y-27632 and C+E. Figure IS illustrates the results of true single-cell cloning experiments performed using H9 cells that were plated as 1 cell/well condition (96-well plates). The treatment with CEPT produced significantly more clones than the treatment with Y-27632 at 10 μM.

The above experiments demonstrated that Trans-ISRIB and Polyamines had a reproducible effect and further improved cell survival, when added to the combination of Chroman 1 and Emricasan.

### Example 8

### Beneficial Effects of CEPT

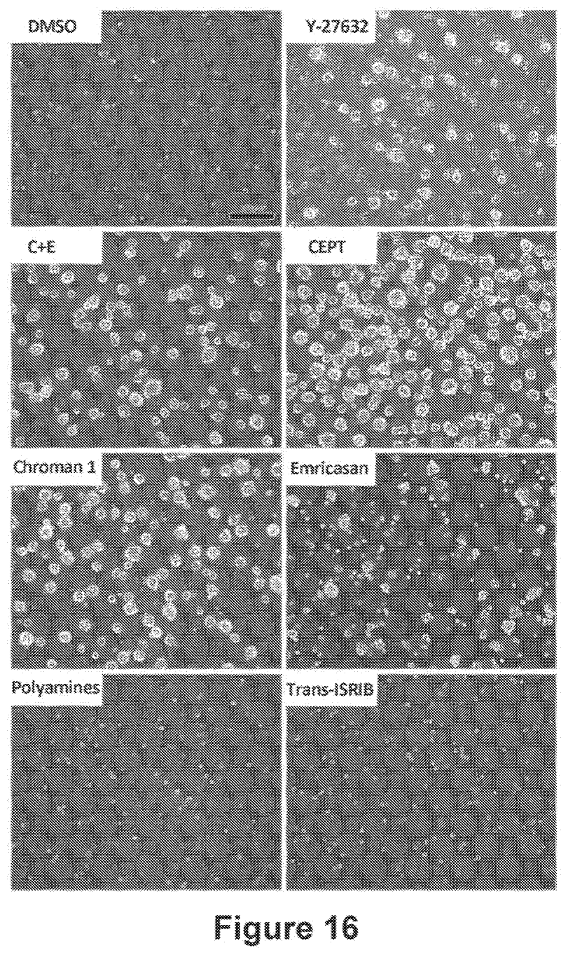

Various experiments were performed showing beneficial effects of CEPT on the survival of hPSCs in culture and various related processes, such as embryoid body and organoid formation and tissue differentiation. Representative experiments are discussed below, with the results illustrated in FIGS. 16-28.

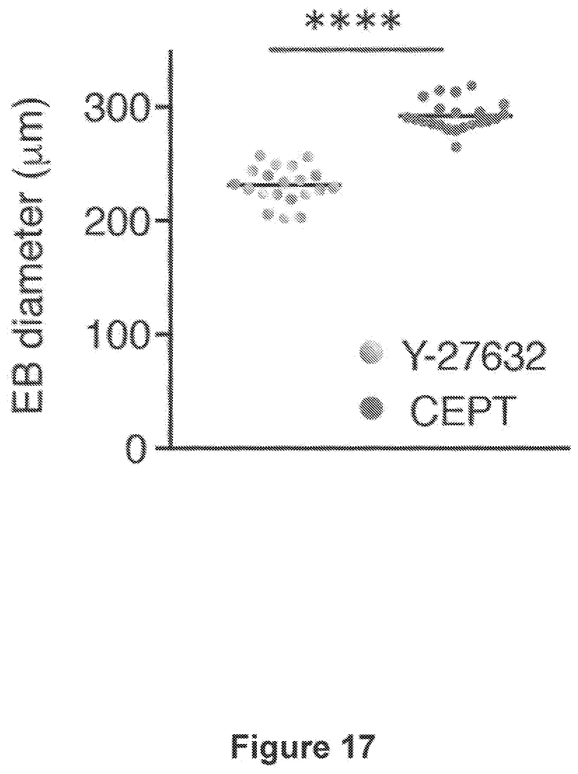

FIG. 16 shows the superiority of CEPT for the outcome of embryoid body formation from H9 cells. FIG. 16 shows representative phase-contrast microscopy images of embryonic bodies formed from 1-19 cells grown in culture media with different supplements, denoted as follows (CEPT—50 nM Chroman 1, 5 μM Emricasan, Polyamines (40 ng/ml, putrescine, 4.5 ng/ml, spermidine, 8 ng/mL spermine), 0.7 μM Trans-ISRIB, C+E—50 nM Chroman 1, 5 μM Emricasan; Chroman 1-50 nM Chroman 1, Emricasan—5 μM Emricasan, Trans-IIRIB—0.7 μM Trans-ISRIB; Polyamines—40 ng/mL putrescine, 4.5 ng/mL spermidine and 8 ng/mL spermine, Y-27632-10 μM). The above notations and concentrations are also used in all the experiments described below. The images were taken 24 hours after cell plating (20,000 cells/cm²). FIG. 17 shows a scatter plot illustrating the quantification of the diameter of single embryoid bodies formed from the cells treated with either Y-27632 or CEPT. To generate single embryoid bodies, 1-19 cells were dissociated with Accutase and plated into AggreWell™ plates (StemCell Technologies, catalog number: 34825) at 5,000 cells/well. The images were taken 24 hours post-plating.

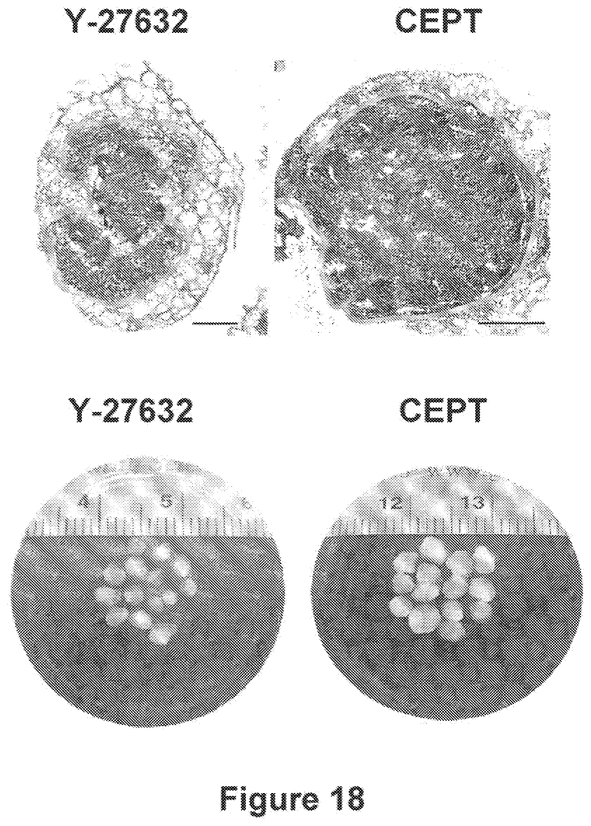

FIG. 18 shows representative images illustrating that CEPT improved cerebral organoid formation, as compared to Y-27632. Cerebral organoids were generated by using a kit obtained from StemCell Technologies (Vancouver, Canada) and iPSCs (LiPSC GR1.1), which were treated with Y-27632 or CEPT for the first 24 hours only. At day 30, organoids were fixed, sectioned, processed for histology and hematoxylin and eosin stain. The results of the histological staining are illustrated in the upper panel. Dark areas in the core of the cerebral organoids show nervous system tissue. Organoid size depended on treatment with Y-27632 or CEPT, which was apparent at day 60. The lower panels illustrate organoid measurements at day 60.

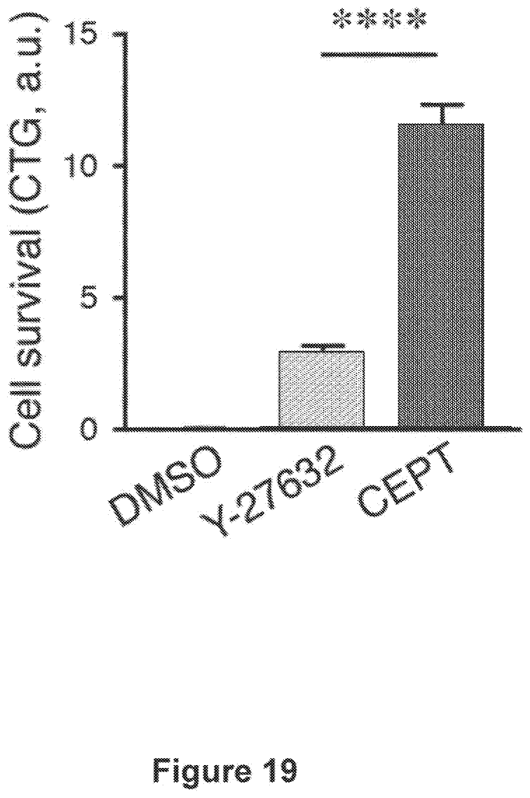

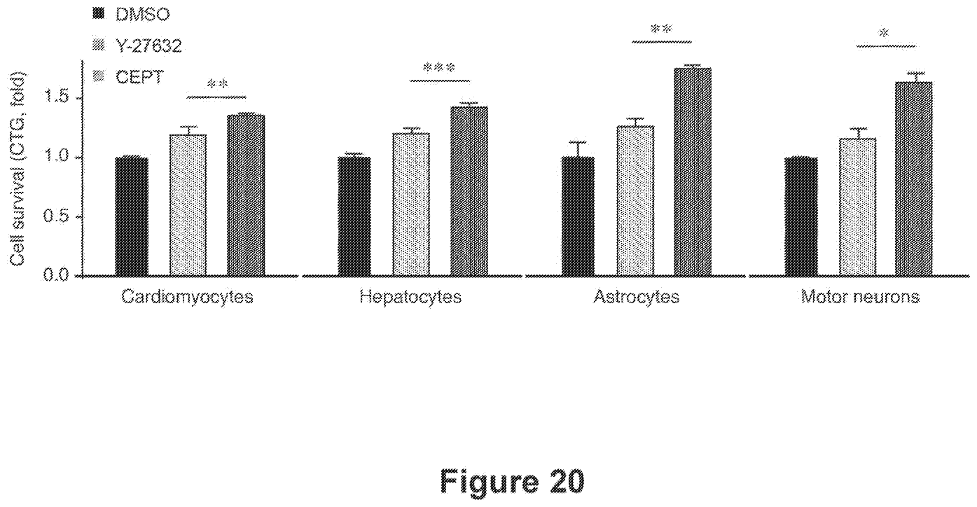

FIG. 19 is a bar graph illustrating improved thawing of that cryopreserved pluripotent stem cells (H9). The cells were thawed and plated in E8 medium in the presence of 0.0001% v/v 0.0001% v/v DMSO, Y-27632 or CEPT. CellTiter Glo® assay was used to quantify live cells 24 hours post-thawing. FIG. 20 is a bar graph illustrating CEPT-improved thawing of various iPSC-derived differentiated cells (all commercially available from Fujifilm Cellular Dynamics International (Madison, Wis.)). Frozen vials of iPSC-derived human cardiomyocytes obtained from Fujifilm Cellular Dynamics International, hepatocytes obtained from Fujifilm Cellular Dynamics, astrocytes differentiated in the laboratory, and motor neurons obtained from Fujifilm Cellular Dynamics International were thawed and treated with 0.0001% v/v DMSO, Y-27632, and CEPT for 24 hours. Cell survival was quantified using the CellTiter Glo assay. The plotted data are mean values ±s.d. (n=3 wells for each group), *P<0.05, **P<0.005, ***P<0.001, one-way ANOVA.

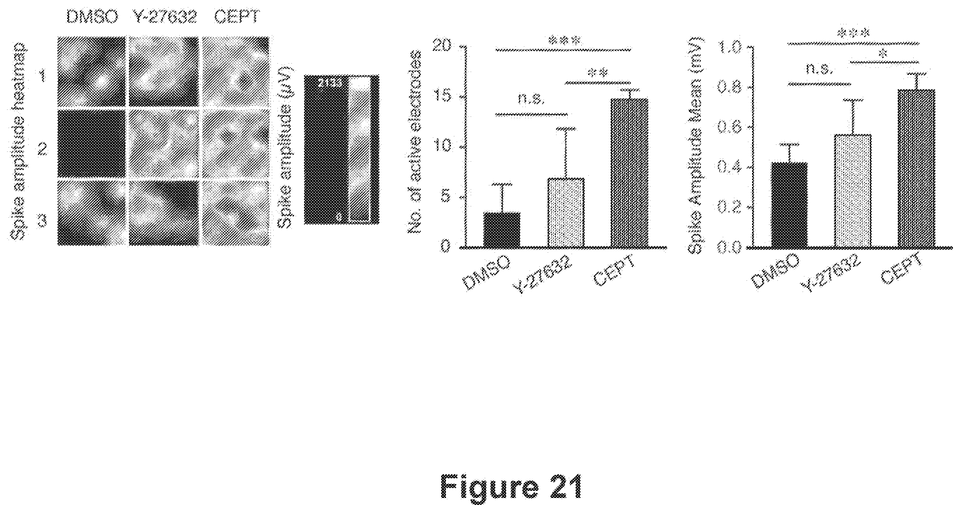

FIG. 21 illustrates the results of electrophysiological characterization of iPSC-derived cardiomyocytes 5 days post-thawing using multi-electrode arrays (Axion Biosystems, Atlanta, Ga.). CEPT treatment for 24 hours was sufficient to improve the recovery from thawing and functional activity of the cardiomyocytes. The data in the middle panel of FIG. 21 represent mean values ±s.d. (n=6 wells for each group), **P=0.0029, ***P=0.0001, one-way ANOVA The data in the right panel of FIG. 21 represent mean±s.d. (n=6 wells for each group), *P=0.0165, ***P=0.0005, one-way ANOVA.

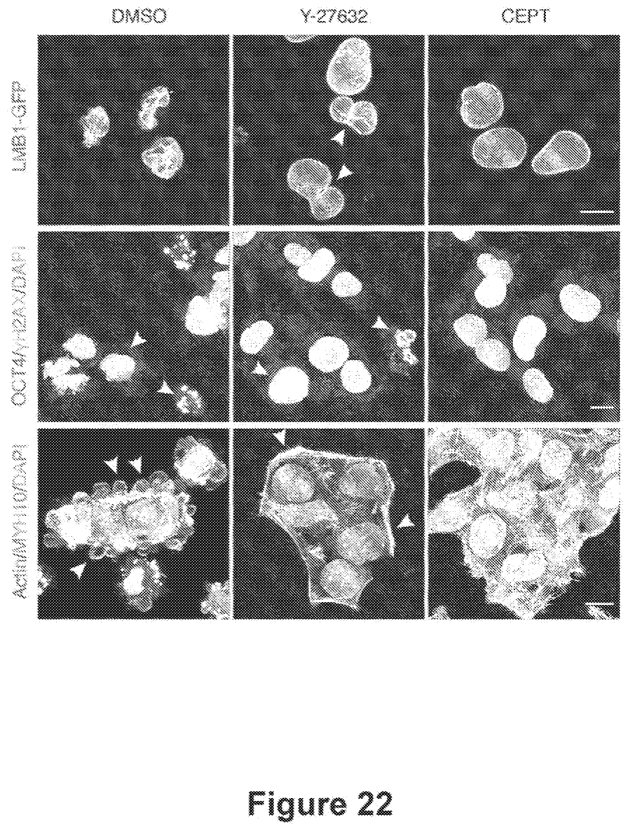

FIG. 22 shows representative microscopic images illustrating CEPT protection of dissociated cells from multiple stress mechanisms. The scale bars shown are 10 μm. Upper panel illustrates confocal microscopic analysis of the lamin B1-GFP iPSC reporter line (Allen Insitute for Cell Science, Seattle, Wash.) displaying dramatic morphological differences in nuclear shape during cell passaging (30 min after plating). Middle panel illustrates that OCT4 expressing cells were immunoreactive for γH2AX when exposed to 0.0001% v/v DMSO and Y-27632 (arrowheads) but not when treated with CEPT (3 hours post-plating). Lower panel illustrates dramatic cytoskeletal differences during cell passaging (3 hours post-plating), as measured by immunocytochemistry against actin and myosin. Stressed cells showed blebbing (white arrowheads) in the presence of 0.0001% v/v DMSO or form prominent actin stress fibers at the colony edge when exposed to Y-27632 (white arrowheads).

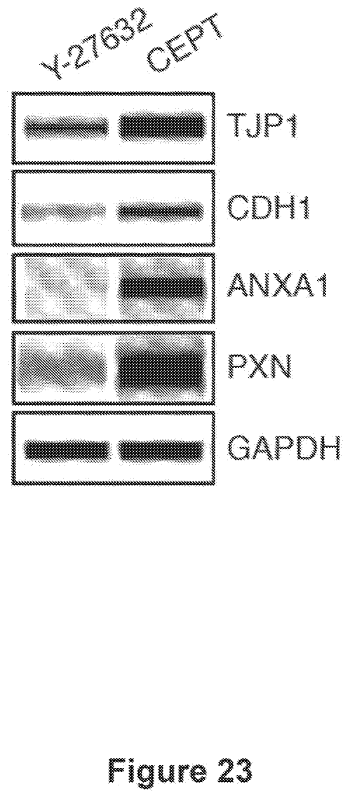

FIG. 23 shows an image of a representative Western blot characterizing hESCs (H9) treated with Y-27632 or CEPT. Several membrane-associated proteins (TJP1, CDH1, ANXA1, PXN) were expressed at higher levels after CEPT treatment (24 hours post-plating). GAPDH was used as a loading control and house-keeping protein control.

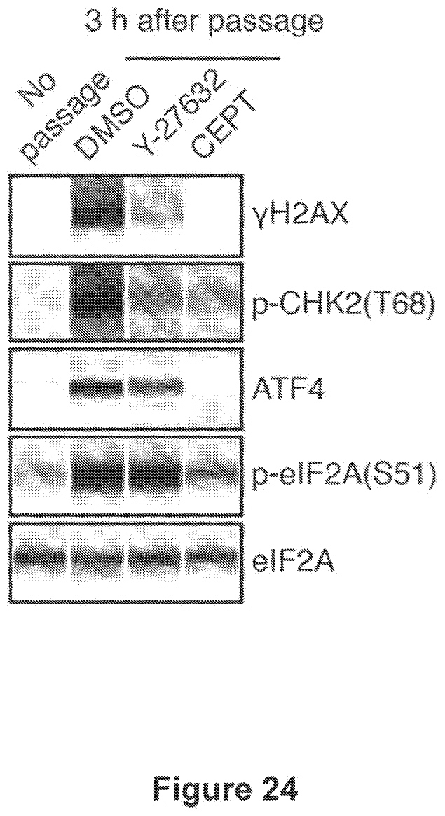

FIG. 24 shows an image of a representative Western blot characterizing hESCs (H9) treated with Y-27632 or CEPT. Strong stress response was observed in the cells treated with 0.0001% v/v DMSO or Y-27632 (3 hours post-passage). The absence of γH2AX (a marker for DNA damage) and ATF4 (a master regulator of cellular stress response) was observed in CEPT-treated cells resembling the control group (no passage). Higher observed levels of phosphorylated CHK2 on threonine 68 indicated cell cycle arrest. While the total expression level of eIF2A remained unaffected, CEPT treatment showed significantly less phosphorylated eIF2A at serine 51. High levels of p-eIF2A(S51) indicated cellular stress and stalled protein synthesis, which occurred in the presence of 0.0001% v/v DMSO or Y-27632.

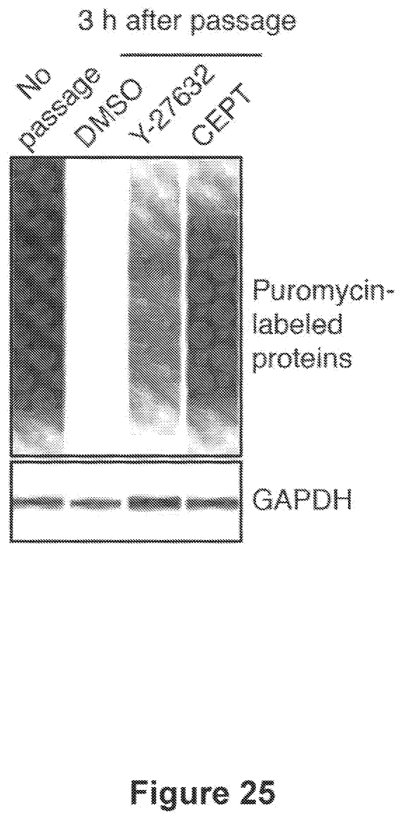

FIG. 25 shows a representative image illustrating the results of puromycin pulse-chase experiment of hESCs (H9) demonstrating that protein synthesis strongly impaired during cell passaging was rescued by CEPT (3 hours post-passage). GAPDH was used as a loading control and house-keeping protein control. To monitor protein synthesis, cells were allowed to adhere for 2 hour and then pulsed with 1 μM puromycin for 1 hour subsequently. Cells were harvested and processed for Western blotting, in which an antibody against puromycin was used (1:50; Sigma-Aldrich, MABE343).

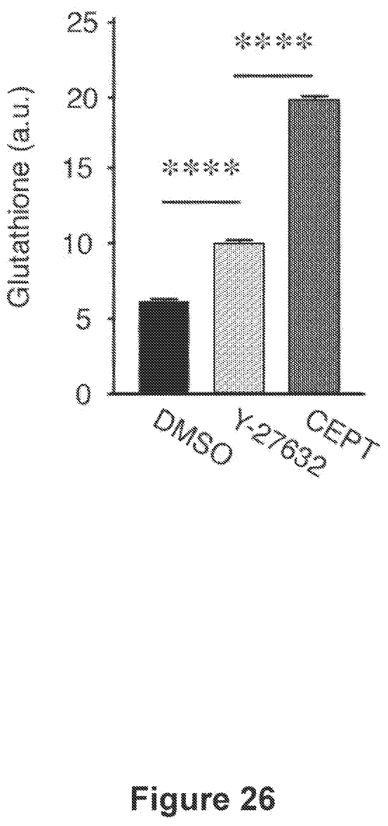

FIG. 26 shows a bar graph illustrating that glutathione levels were significantly higher in hESCs (H9) passaged with CEPT as compared to 0.0001% v/v DMSO and Y-27632 (3 hours post-plating). Glutathione is an important intracellular antioxidant, and lower levels of glutathione indicated oxidative stress.

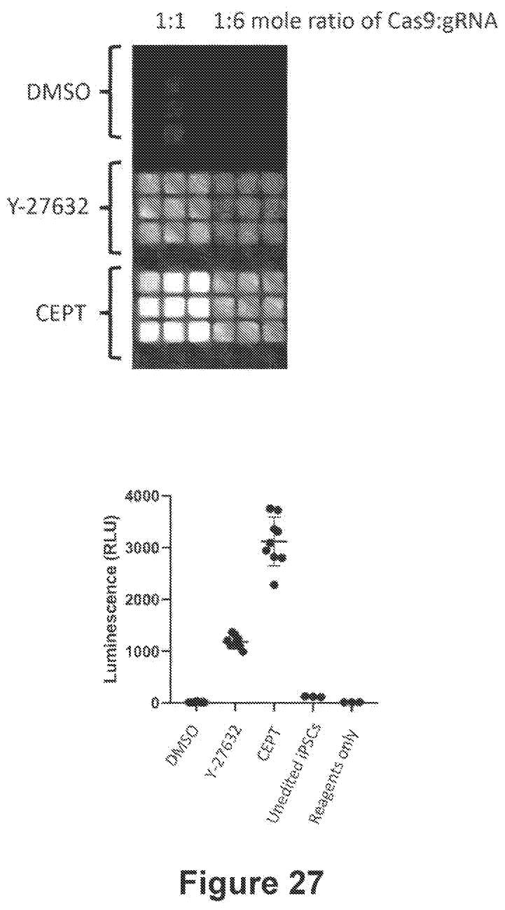

FIG. 27 shows that CEPT improved genome editing efficiency. A reporter iPSC line was generated by CRISPR/Cas9 Knock-in according to the methods described in Schwinn el al., “CRISPR-mediated tagging of endogenous proteins with a luminescent peptide” ACS Chemical Biology 13:467-474 (2018). A luminescent peptide was introduced into the OCT4 gene of an iPSC line, and the signal was detected by using the Nano-Glo™ HiBiT Detection System (Promega, Madison, Wis.). CEPT treatment during gene editing resulted in increased gene editing efficiency, as compared to Y-27632 treatment. The image in the upper panel shows luminescence intensities, which were measured 24 hours after transfecting Cas9 and the guide RNA into iPSCs. The scatter plot in the lower panel illustrates the quantification of the luminescence signal. Controls included “Unedited iPSCs” and “Reagents only.”

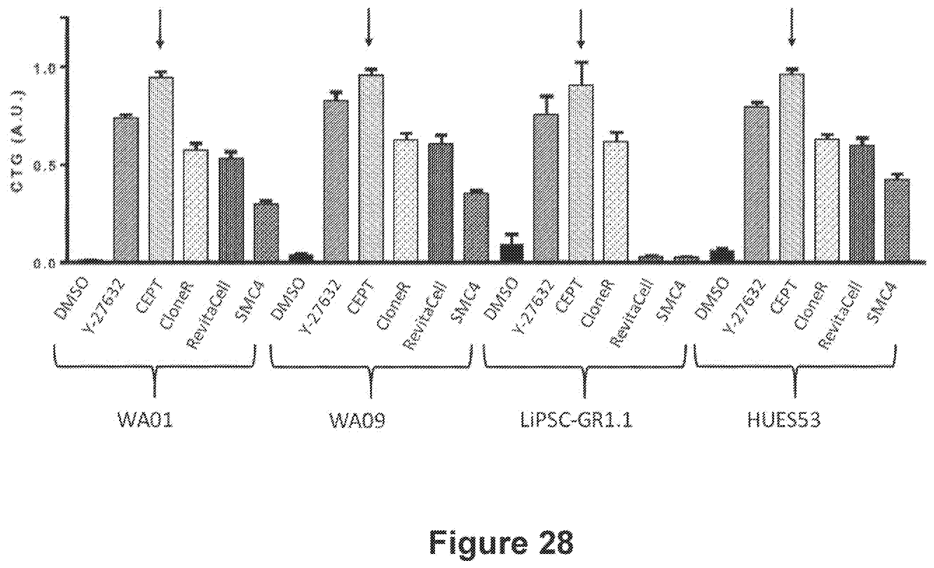

FIG. 28 shows a bar graph comparing the survival of human pluripotent stem cells in the presence of various reagents and CEPT. Human embryonic stem cell lines (WA01, WA09, HUES53) and iPSCs (LiPSC-GR1.1) were dissociated by Accutase (Thermo Fisher Scientific) and equal numbers of cells were plated into vitronectin-coated 384-well plates in E8 medium. CellTiter Glo® assay was used to quantify viability of cells at 24 hours post-plating. Despite the well-known cell-line-cell line variability, in each case CEPT was shown to be superior to Y-27632 and the commercially available reagents CloneR™ (StemCell Technologies), RevitaCell™ (Thermo Fisher Scientific) and StemBoost™ Reprogramming Cocktail SMC4 (BioVision).

All patents, patent applications, publications, and abstracts cited above are incorporated herein by reference in their entirety. Various embodiments of the invention have been described in fulfillment of the various objectives of the invention. It should be recognized that these embodiments are merely illustrative of the principles of the present invention. Numerous modifications and adaptations thereof will be readily apparent to those of skill in the art without departing from the spirit and scope of the invention as defined in the following claims.

TABLE 1
Active compounds identified by qHTS

| | | PubChem | CAS | | | | |
|---|---|---|---|---|---|---|---|
| | | Substance | Registry | | AC₅₀⁵ | | |
| Compound | SMILES¹ | Identifier² | Number³ | CC⁴ | (μM) | Efficacy⁶ | Max⁶ |
| GSK-269962A | CC[N]1C2═C(C═NC(═C | 137275847 | 850664-21-0 | 2 | 11.77 | 106 | 133.3 |
| | 2)OC3═CC(═CC═C3)NC | | | | | | |
| | (═O)C4═CC═C(OCCN5C | | | | | | |
| | COCC5)C═C4)N═C1C6═ | | | | | | |
| | NON═C6N | | | | | | |
| GSK-429286A | CC/1═C(/C(CC(═O)N1)C | 174006479 | 864082-47-3 | 1 | 3.317 | 126 | 129.3 |
| | 2═CC═C(C═C2)C(F)(F)F) | | | | | | |
| | C(═O)NC3═C(F)C═C4 | | | | | | |
| | [NH]N═CC4═C3 | | | | | | |
| Y-39983 | C[C@@H](N)C1═CC═C | | 173897-44-4 | 1 | 0.369 | 121 | 127 |
| | (C═C1)C(═O)NC2═C3C═ | | | | | | |
| | C[NH]C3═NC═C2 | | | | | | |
| 1-(3- | COC1═CC(═CC═C1)CN | | 1342276-76-9 | 1 | 0.147 | 121 | 123.6 |
| methoxybenzyl)-3- | C(═O)NC2═NC(═CS2) | | | | | | |
| (4-(pyridin-4-yl) | C3═CC═NC═C3 | | | | | | |
| thiazol-2-yl)urea | | | | | | | |
| SR-3677 | CN(C)CCOC1═CC(═C | | 1072959-67-1 | 1 | 0.585 | 118 | 122 |
| | C═C1NC(═O)C2COC3═C | | | | | | |
| | C═CC═C3O2)C4═ | | | | | | |
| | C[NH]N═C4 | | | | | | |
| SR-3677 | CN(C)CCOC1═CC(═C | 174006422 | 1072959-67-1 | 1 | 0.662 | 115 | 121.4 |
| | C═C1NC(═O)C2COC3═C | | | | | | |
| | C═CC═C3O2)C4═ | | | | | | |
| | C[NH]N═C4 | | | | | | |
| GSK-25 | CC\1═C(C(\N═C(/N1)C2═ | 174006433 | 874119-56-9 | 1 | 1.49 | 118 | 121.1 |
| | CC(═NC═C2)C1)C3═C | | | | | | |
| | (F)C═C(C1)C═C3)/C(═O) | | | | | | |
| | NC4═C(F)C═C5[NH]N═ | | | | | | |
| | CC5═C4 | | | | | | |
| Glycyl-H-1152 | C[C@H]1CN(CCCN1 | 174006415 | 913844-45-8 | 1 | 3.317 | 120 | 120.9 |
| | [S+]([O−])(═O) | | | | | | |
| | C2═CC═CC3═C2C | | | | | | |
| | (═CN═C3)C)C(═O)CN | | | | | | |
| AT-7867 | C1C1═CC═C(O═C1)C2(C | 174006525 | 857531-00-1 | 1 | 1.49 | 92.2 | 119.5 |
| | CNCC2)C3═CC═C(C═ | | | | | | |
| | C3)C4═C[NH]N═C4 | | | | | | |
| AT-7867 | C1C1═CC═C(O═C1)C2(C | | 857531-00-1 | 1 | 0.521 | 104 | 118.4 |
| | CNCC2)C3═CC═C(C═ | | | | | | |
| | C3)C4═C[NH]N═C4 | | | | | | |
| AS 1892802 | OC[C@@H](NC(═O)NC1═ | | 928320-12-1 | 1 | 1.309 | 115 | 117.5 |
| | CC═C(C═C1)C2═CC═ | | | | | | |
| | NC═C2)C3═CC═CC═C3 | | | | | | |
| Y-39983 | C[C@@H](N)C1═CC═C | 174006412 | 173897-44-4 | 1 | 0.743 | 113 | 116.8 |
| | (C═C1)C(═O)NC2═C3C═ | | | | | | |
| | C[NH]C3═NC═C2 | | | | | | |
| SAR407899 | O═C1N\C═C/C2═CC(═C | | 923262-96-8 | 1 | 2.931 | 112 | 116.3 |
| (hydrochloride) | C═C12)OC3CCNCC3 | | | | | | |
| Baricitinib | CC[S+]([O−]) | 174006690 | 1187594-9-7 | 3 | 37.22 | 147 | 114.7 |
| | (═O)N1CC(CC#N)(C1) | | | | | | |
| | [N]2C═C(C═N2)C3═ | | | | | | |
| | NC═NC4═C3C═C[NH]4 | | | | | | |
| GSK-25 | CC\1═C(C(\N═C(/N1)C2═ | | (874119-56-9) | 1 | 0.185 | 113 | 113.3 |
| | CC(═NC═C2)C1)C3═C | | | | | | |
| | (F)C═C(C1)C═C3)/C(═O) | | | | | | |
| | NC4═C(F)C═C5[NH] | | | | | | |
| | N═CC5═C4 | | | | | | |
| Glycyl-H-1152 | C[C@H]1CN(CCCN1 | | [913844-45-8] | 1 | 0.414 | 96.9 | 112.3 |
| | [S+]([O−])(═O) | | | | | | |
| | C2═CC═CC3═C2C | | | | | | |
| | (═CN═C3)C)C(═O)CN | | | | | | |
| Y-27632 | C[C@@H](N)C1CC | 90340876 | 331752-47-7 | 1 | 1.321 | 102 | 112.1 |
| | [C@@H](CC1)C(═O) | | | | | | |
| | NC2═CC═NC═C2 | | | | | | |
| SB-747651-A | CC[N]1C(═NC2═CN═CC | 174007095 | 607372-46-3 | 1 | 2.957 | 68.7 | 111.1 |
| | (═C12)CNC3CCNCC3) | | | | | | |
| | C4═NON═C4N | | | | | | |
| Chroman 1 | COC1═CC═C2OC[C@H] | | 1273579-40-0 | 1 | 0.29 | 108 | 110.1 |
| | (CC2═C1)C(═O)NC3═ | | | | | | |
| | CC═C(C═C3OCCN(C)C) | | | | | | |
| | C4═C[NH]N═C4 | | | | | | |
| OXA 06 | FC1═CC═CC═C1CNCC2═ | | | 1 | 0.736 | 110 | 108.2 |
| dihydrochloride | CC═C(C═C2)C3═C4C═ | | | | | | |
| | C[NH]C4═NC═C3 | | | | | | |
| (−)-Blebbistatin | CC1═CC═C2\N═C/3N(C | | 856925-71-8 | 1 | 2.931 | 96 | 107.8 |
| | C[C@@]3(O)C(═O)C2═ | | | | | | |
| | C1)C4═CC═CC═C4 | | | | | | |
| Fasudil | [O−][S+](═O) | 124883255 | 105628-7-7 | 3 | 33.17 | 127 | 107.2 |
| hydrochloride | (N1CCCNCC1) | | | | | | |
| | C2═C3C═CN═ | | | | | | |
| | CC3═CC═C2 | | | | | | |
| CCT-128930 | NC1(CCN(CC1)C2═ | 174006521 | 885499-61-6 | 3 | 11.77 | 133 | 106.8 |
| | C3C═C[NH]C3═NC═N2) | | | | | | |
| | CC4═CC═C(C1)C═C4 | | | | | | |
| ROCK inhibitor | CC1═C[NH]C2═NC═CC | | 867017-68-3 | 1 | 0.21 | 59.8 | 103.2 |
| | (═C12)OC3═C(F)C═C | | | | | | |
| | (NC4═NC(═NC(═C4) | | | | | | |
| | C1)N)C═C3F | | | | | | |
| RKI-1447 | OC1═CC(═CC═C1)CNC | | 1342278-01-6 | 1 | 0.083 | 106 | 103.2 |
| | (═O)NC2═NC(═CS2) | | | | | | |
| | C3═CC═NC═C3 | | | | | | |
| Baricitinib | CC[S+]([O−](═O) | | 1187594-9-7 | 2 | 8.26 | 114 | 102.7 |
| | N1CC(CC#N)(C1) | | | | | | |
| | [N]2C═C(C═N2)C3═NC═ | | | | | | |
| | NC4═C3C═C[NH]4 | | | | | | |
| KX1-004 | OC1═CC(═CC═C1)CNC | | 518058-84-9 | 1 | 3.289 | 103 | 102.2 |
| | (═O)C2═CC3═CC | | | | | | |
| | (═CC═C3[NH]2)F | | | | | | |
| 1-05- | CC1CN(CCN1)[S+]([O−]) | 90341486 | | 3 | 20.6 | 102 | 100.6 |
| Isoquinolinylstil | (═O)C2═C3C═CN═ | | | | | | |
| fonyI)-3- | CC3═CC═C2 | | | | | | |
| methylpiperazine | | | | | | | |
| dihydroehloride | | | | | | | |
| Y-27632 | C[C@@H](N)C1CC | 90340876 | 331752-47-7 | 2 | 7.308 | 96.2 | 95.49 |
| | [C@@H](CC1)C(═O) | | | | | | |
| | NC2═CC═NC═C2 | | | | | | |
| Oclacitinib | CN[S+]([O−](═O) | | 1208319-26-9 | 3 | 20.75 | 99.9 | 93.79 |
| (PF-03394197) | C[C@H]1CC[C@H] | | | | | | |
| | (CC1)N(C)C2═C3C═ | | | | | | |
| | C[NH]C3═NC═N2 | | | | | | |
| Q-VD-OPh | CC(C)C(NC(═O)C1═NC | | 1135695-98-5 | 1 | 1.167 | 85.9 | 93.54 |
| hydrate | 2═CC═CC═C2C═C1)C | | | | | | |
| | (═O)NC(CC(O)═O)C(═O)C | | | | | | |
| | OC3═C(F)C═CC═C3F | | | | | | |
| CCT-241533 | COC1═CC2═NC(═NC(═ | 174006969 | 1262849-73-9 | 3 | 11.77 | 118 | 92.8 |
| | C2C═C1OC)N[C@@H]3 | | | | | | |
| | CNC[C@H]3C(C)(C)O) | | | | | | |
| | C4═CC(═CC═C4O)F | | | | | | |
| (−)-Blebbistatin | CC1═CC═C2\N═C/3N(C | 26753178 | 674289-55-5 | 3 | 33.17 | 105 | 90.71 |
| | C[C@@H]3(O)C(═O)C2═ | | | | | | |
| | C1)C4═CC═CC═C4 | | | | | | |
| AT-13148 | NC[C@@H](O)(C1═CC═C | | 1056901-62-2 | 1 | 0.104 | 64.5 | 89.77 |
| | (C1)C═C1)C2═CC═C(C═ | | | | | | |
| | C2)C3═C[NH]N═C3 | | | | | | |
| RO495 | CC1═NC(═NC(═C1)NC2═ | 137276044 | 1258296-60-4 | 1 | 4.686 | 73.1 | 89.28 |
| | CC(═CC═N2)NC(═O) | | | | | | |
| | C3═C(C1)C═CC═C3C1)N | | | | | | |
| Thiazovivin | O═C(NCC1═CC═CC═C1) | | 1226056-71-8 | 1 | 0.414 | 80.6 | 88.3 |
| | C2═CSC(═N2)NC3═CC | | | | | | |
| | ═NC═N3 | | | | | | |
| HA-100 | [O−][S+](═O)(N1CCNCC1) | 90341528 | | 2 | 7.308 | 88.9 | 88.17 |
| | C2═C3C═CC═NC3═CC═C2 | | | | | | |
| Ruxolitinib, | N#CCC(C1CCCC1)[N]2 | | 941678-49-5 | 3 | 10.4 | 48 | 87.05 |
| Freebase | C═C(C═N2)C3═C4C═ | | | | | | |
| | C[NH]C4═NC═N3 | | | | | | |
| 1-(5- | CC1CNCCN1[S+]([O−]) | 90341387 | | 3 | 4.611 | 92.2 | 85.52 |
| Isoquinolinylsul | (═O)C2═C3C═CN═ | | | | | | |
| fonyl)-2- | CC3═CC═C2 | | | | | | |
| methylpipenvine | | | | | | | |
| dihydrochloride | | | | | | | |
| Pinacidil | CC(N\C(NC1═CC═NC═ | 90341497 | | 3 | 21.11 | 86.4 | 83.12 |
| | C1)═N/C#N)C(C)(C)C | | | | | | |
| Ruxolitinib | N#CC[C@H](C1CCCC1) | | 1092939-17-7 | 2 | 8.26 | 89.8 | 81.17 |
| | [N]2C═C(C═N2)C3═NC═ | | | | | | |
| | NC4═C3C═C[NH]4 | | | | | | |
| Momelotinib | O═C(NCC#N)C1═CC═C | 137273866 | 1036634-68-4 | 1 | 0.935 | 38.7 | 79.24 |
| | (C═C1)C2═NC(═NC═C2) | | | | | | |
| | NC3═CC═C(C═C3) | | | | | | |
| | N4CCOCC4 | | | | | | |
| SB-747651-A | CC[N]1C(═NC2═CN═CC | | N.A. | 1 | 0.585 | 35.5 | 78.33 |
| | (═C12)CNC3CCNCC3)C | | | | | | |
| | 4═NON═C4N | | | | | | |
| Emricasan | C[C@H](NC(═O)C(═O) | 174006947 | 254750-02-2 | 3 | 33.17 | 95.8 | 76.39 |
| | NC1═CC═CC═C1C(C)(C) | | | | | | |
| | C)C(═O)N[C@@H](CC | | | | | | |
| | (O)═O)C(═O)COC2═C(F) | | | | | | |
| | C(═CC(═C2F)F)F | | | | | | |
| KRIBB11 | CNC1═NC(═C(C═C1) | | [342639-96-7] | 1 | 0.585 | 62.1 | 72.62 |
| | [N+])═O)NC2═CC═C3 | | | | | | |
| | [NH]═CC3═C2 | | | | | | |
| Fluoxetine | CNCCC(OC1═CC═C | 90341619 | | 4 | | 0 | 70.37 |
| hydrochloride | (C═C1)C(F)(F)F)C2═ | | | | | | |
| | CC═CC═C2 | | | | | | |
| STF-083010 | OC1═CC═C2C═CC═ | 137273983 | 307343-71-1 | 2 | 26.35 | 82.4 | 66.46 |
| | CC2═C1/C═N/[S+]([O−]) | | | | | | |
| | (═O)C3═CC═CS3 | | | | | | |
| Ruxolitinib | N#CC[C@H](C1CCCC1) | 174006683 | 941678-49-5 | 3 | 11.77 | 80.4 | 63.7 |
| | [N]2C═C(C═N2)C3═NC═ | | | | | | |
| | NC4═C3C═C[NF]4 | | | | | | |
| dBETI | CC1═C(C)C\2═C(S1)[N] | | 1799711-21-9 | −1 | 0.004 | −67.9 | 62.14 |
| | 3C(═NN═C3[C@@H](C | | | | | | |
| | C(═O)NCCCCNC(═O)C | | | | | | |
| | OC4═C5C(═O)N([C@H] | | | | | | |
| | 6CCC(═O)NC6═O)C(═O) | | | | | | |
| | C5═CC═C4)\N═C2C7═C | | | | | | |
| | C═C(C1)C═C7)C | | | | | | |
| Rhodblock 6 | O═C(NC1═CC2═C([NH] | | 886625-06-5 | 3 | 23.28 | 67.9 | 61.61 |
| | N═C2)C═C1)C3CCC3 | | | | | | |
| NCGC00244250 | CN(C)C1═CC═C(NC2═N | 174006689 | 1261496-45-0 | 4 | 6.561 | 23.7 | 58.27 |
| | [N](C(═N2)N)C(═O)C3═ | | | | | | |
| | C(F)C═CC═C3F)C═C1 | | | | | | |
| AMG-Tie2-1 | CNC1═NC(═CC═N1)C2═ | 174006749 | 870223-96-4 | 2 | 18.65 | 54.5 | 57.84 |
| | CC═CN═C2OC3═CC(═C | | | | | | |
| | C═C3C)C(═O)NC4═CC═ | | | | | | |
| | CC(═C4)C(F)(F)F | | | | | | |
| Ingenol | C\C═C(C)/C(═O)O[C@H] | 174006398 | 75367-37-2 | 1 | 0.118 | 41.4 | 57.64 |
| mebutate | 1C(═C/[C@@]23[C@H] | | | | | | |
| | (C)C[C@@H]4[C@H] | | | | | | |
| | ([C@H](\C═C(CO)/[C@@H] | | | | | | |
| | (O)[C@]12O)C3═ | | | | | | |
| | O)C4(C)C\C | | | | | | |
| TG-101348 | CC1═C(NC2═CC(═ | 137275863 | 936091-26-8 | 3 | 33.17 | 67.3 | 55.15 |
| | CC═C2)[S+]([O−]) | | | | | | |
| | (═O)NC(C)(C)C)N═C | | | | | | |
| | (NC3═CC═C(OCCN4CC | | | | | | |
| | CC4)C═C3)N═C1 | | | | | | |
| Momelotinib | O═C(NCC#N)C1═CC═C | | 1056634-68-4 | 4 | | 0 | 53.99 |
| | (C═C1)C2═NC(═NC═C2) | | | | | | |
| | NC3═CC═C(C═C3) | | | | | | |
| | N4CCOCC4 | | | | | | |
| LX-7101 | CN(C)C(═O)OC1═CC═C | | 1192189-69-7 | 4 | 2.328 | 21.6 | 53.74 |
| | C(═C1)NC(═O)C2(CN)C | | | | | | |
| | CN(CC2)C3═C4C(═C[NH] | | | | | | |
| | C4═NC═N3)C | | | | | | |
| Lestaurtinib | C[C@@]12O[C@@H](C | 174006757 | 111358-88-4 | 4 | 1.177 | 7.59 | 52.98 |
| | [C@]1(O)CO[N]3C4═C | | | | | | |
| | (C═CC═C4)C5═C6C(═O) | | | | | | |
| | NCC6═C7C8═C(C═CC═ | | | | | | |
| | C8)[N]2C7═C35 | | | | | | |
| SB-415286 | OC1═C(C1)C═C(N\C2═C | 174007074 | 264218-23-7 | 3 | 33.17 | 65.1 | 52.48 |
| | (\C(═O)NC2═O)C3═C | | | | | | |
| | (C═CC═C3)[N+] | | | | | | |
| | ([O−])═O)C═C1 | | | | | | |
| (−)-Indolactam V | CC(C)[C@@H]1N(C) | 174007218 | 90365-57-4 | 1 | 0.833 | 45.5 | 51.21 |
| | C2═C3C═C[NH]C3═CC═ | | | | | | |
| | C2)C[C@@H](CO) | | | | | | |
| | NCI═O | | | | | | |
| HA-1004 | NC(═N)NCCNO[S+]([O−]) | 90340739 | | 1 | 5.174 | 50 | 50.05 |
| hydrochloride | C1═C2C═CN═CC2═ | | | | | | |
| | CC═C1 | | | | | | |
| Go-6976 | C[N]1C2═C(C═CC═C2)C | 174006399 | 136194-77-9 | 2 | 4.686 | 37.3 | 49.06 |
| | 3═C4C(═O)NCC4═C5C6═ | | | | | | |
| | C(C═CC═C6)[N] | | | | | | |
| | (CCC#N)C5═C13 | | | | | | |
| Phorbol | CCCCCCCCCCCCCC(═ | 90341751 | | 1 | 0.21 | 36.8 | 48.49 |
| 12-myristate | O)O[C@@H]1[C@@H] | | | | | | |
| 13-acetate | (C)[C@@]2(O)[C@@H] | | | | | | |
| | (\C═C(CO)/C[C@]3(O) | | | | | | |
| | [C@H]2\C═C(C)/C3═O) | | | | | | |
| | [C@@H]4C(C)(C)[C@]14 | | | | | | |
| | OC(C)═O | | | | | | |
| PF-956980 | C[C@@H]1CCN(C | | 1262832-74-5 | 3 | 23.28 | 53 | 47.75 |
| | [C@@H]1N(C)C2═C3C═ | | | | | | |
| | C[NH]C3═NC═N2)C(═O) | | | | | | |
| | N4CCCC4 | | | | | | |
| CK-636 | CC1═C(CCNC(═O) | | 442632-72-6 | 4 | | 0 | 47.74 |
| | C2═CC═CS2)C3═ | | | | | | |
| | CC═CC═C3[NH]1 | | | | | | |
| AKT Kinase | CC[N]1C(═NC2═C1C(═ | | 842148-40-7 | 4 | | 0 | 46.89 |
| Inhibitor | CN═C2C#CCO)OCCCN) | | | | | | |
| | C3═NON═C3N | | | | | | |
| NU-6102 | N[S+]([O−])(═O) | 174006986 | 444722-95-6 | 3 | 37.22 | 57.9 | 46.11 |
| | C1═CC═C(NC2═NC3═ | | | | | | |
| | C([NH]C═N3)C(═N2) | | | | | | |
| | OCC4CCCCC4)C═C1 | | | | | | |
| PF-06447475 | N#CC1═CC═CC(═C1) | | 1527473-33-1 | 3 | 16.48 | 44.6 | 42.8 |
| | C2═C[NH]C3═NC═NC | | | | | | |
| | (═C23)N4CCOCC4 | | | | | | |
| BMS-265246 | CCCCOC1═C2C═N[NH] | 174007097 | 582315-72-8 | 1 | 1.177 | 33.9 | 42.75 |
| | C2═NC═C1C(═O)C3═ | | | | | | |
| | (CF)C═C(C)C═C3F | | | | | | |
| 1,5- | OC1═CC═CC2═ | 90340598 | | 3 | 18.36 | 42.2 | 41.01 |
| Isoquinolinediol | C(O)N═CC═C12 | | | | | | |
| NCGC00244250 | CN(C)C1═CC═C(NC2═N | 174006689 | 1261496-45-0 | 1 | 8.333 | 39.3 | 40.04 |
| | [N](C(═N2)N)C(═O)C3═ | | | | | | |
| | C(F)C═CC═C3F)C═C1 | | | | | | |
| KW-2449 | O═C(N1CCNCC1)C2═C | 174006554 | 1000669-72-6 | 3 | 33.17 | 44.5 | 40.04 |
| | C═C(C═C2)/C═C/C3═N | | | | | | |
| | [NH]C4═C3C═CC═C4 | | | | | | |
| A-443654 | CC1═N[NH]C2═C1C═C | 174006490 | 552325-16-3 | 4 | | 0 | 39.77 |
| | (C═C2)C3═CN═CC(═C3) | | | | | | |
| | OC[C@@H](N)CC4═C | | | | | | |
| | [NH]C5═C4C═CC═C5 | | | | | | |
| Cerdulatinib | CC[S+]([O−]) | | 1198300-79-6 | 1 | 2.931 | 33.6 | 38.99 |
| | (═O)N1CCN(CC1)C2═ | | | | | | |
| | CC═C(NC3═NC(═C | | | | | | |
| | (C═N3)C(N)═O) | | | | | | |
| | NC4CC4)C═C2 | | | | | | |
| NCGC00384798-01 | CC1(C)CC[C@@]2(CC | | | 4 | | 0 | 38.81 |
| | [C@]3(C)\C(═C/CC4[C@@] | | | | | | |
| | 5(C)CC[C@H](O[C@@H] | | | | | | |
| | 6OC[C@@H](O)[C@H] | | | | | | |
| | (O)[C@H]6O)[C@] | | | | | | |
| | (C)(C5CC[C@@]34C) | | | | | | |
| | C(O)═O)[C@@H]2C1)C | | | | | | |
| | (═O)O[C@@H]7O[C@H] | | | | | | |
| | (CO[C@@H]8O[C@H] | | | | | | |
| | (CO)[C@@H](O)[C@H] | | | | | | |
| | (O)[C@H]8O[C@@H]9O | | | | | | |
| | [C@H](CO)[C@H](O) | | | | | | |
| | [C@H](O)[C@H]9O) | | | | | | |
| | [C@H](O)[C@H](O[C@@H] | | | | | | |
| | %10O[C@H](CO)[C@H] | | | | | | |
| | (O)[C@H](O)[C@H] | | | | | | |
| | %10O)[C@H]7O | | | | | | |
| PPY A | COC1═C(C═CC═C1)C2═ | | [875634-01-8] | 3 | 2.931 | 41.9 | 37.42 |
| | C[NH]C3═NC═C(C═C23) | | | | | | |
| | C4═CC(═CN═C4)C(═O) | | | | | | |
| | N(C)C | | | | | | |
| CP-673451 | COCCOC1═CC═C2[N] | 174006575 | 343787-29-1 | 4 | | 0 | 35.65 |
| | (C═NC2═C1)C3═NC4═C | | | | | | |
| | (C═CC═C4C═C3)N5CCC | | | | | | |
| | (N)CC5 | | | | | | |
| SB-203580 | C[S+]([O−]) | 124886984 | 152121-47-6 | 3 | 15.21 | 37.4 | 35.56 |
| | C1═CC═C(C═C1)C2═N | | | | | | |
| | C(═C([NH]2)C3═CC═NC═ | | | | | | |
| | C3)C4═CC═C(F)C═C4 | | | | | | |
| Sotrastaurin | CN1CCN(CC1)C2═NC3═ | 137275939 | 425637-18-9 | 2 | 16.63 | 36.5 | 35.9 |
| | CC═CC═C3C(═N2)C4C | | | | | | |
| | (C(═O)NC4═O)C5═C | | | | | | |
| | [NH]C6═CC═CC═C56 | | | | | | |
| NU-6102 | N[S+]([O−]) | | 444722-95-6 | 3 | 5.212 | 43.8 | 34.8 |
| | (═O)C1═CC═C(NC2═N | | | | | | |
| | C3═C([NH]C═N3)C(═N2) | | | | | | |
| | OCC4CCCCC4)C═C1 | | | | | | |
| CCT-241533 | COC1═CC2═NC(═NC(═ | | 1431697-96-9 | 3 | 5.212 | 43.3 | 34.35 |
| | C2C═C1OC)N[C@@H]3 | | | | | | |
| | CNC[C@H]3C(C)(C)O)C | | | | | | |
| | 4═CC(═CC═C4O)F | | | | | | |
| TETRAC | OC(═O)CC1═CC(═C | | 67-30-1 | 1 | 14.69 | 35.8 | 34.23 |
| | (OC2═CC(═C(O)C(═C2) | | | | | | |
| | 1)1)C(═C1)1)1 | | | | | | |
| Cytochalasin B | C[C@@H]1CCC[C@@H] | | 14930-96-2 | 3 | 7.495 | 43.2 | 14.14 |
| | (O)/C═C/C(═O)O[C@] | | | | | | |
| | 23[C@@H](\C═C\C1) | | | | | | |
| | [C@H](O)C(═C)[C@@H] | | | | | | |
| | (C)[C@H]2[C@H](CC4═ | | | | | | |
| | CC═CC═C4)NC3═O | | | | | | |
| Decernotinib | CC[C@@](C)(NC1═CC═ | | 944842-54-0 | 1 | 2.931 | 30.1 | 34.01 |
| | NC(═N1)C2═C[NH]C3═ | | | | | | |
| | C2C═CC═N3)C(═O) | | | | | | |
| | NCC(F)(F)F | | | | | | |
| NCGC00347879-02 | CC(C)CC(═O)OC1O\C═ | | | 4 | | 0 | 30.87 |
| | C(\CO[C@@H]2O[C@H] | | | | | | |
| | (CO)[C@@H](O)[C@H] | | | | | | |
| | (O)[C@H]2O)C3CC(O) | | | | | | |
| | C(O)(CO)C13 | | | | | | |
| XL-19 | O═C(NC1═CC═C(C═C1) | | 945755-56-6 | 3 | 23.28 | 35.5 | 29.98 |
| | C2═NC(═NC═C2)NC3═C | | | | | | |
| | C═C(C═C3)N4CCOCC4) | | | | | | |
| | C5CCCN5 | | | | | | |
| IKK epsilon-IN-1 | N#CC1═CC(═CC═C1OC | | 1292310-49-6 | 2 | 14.69 | 30.6 | 28.74 |
| | 2CCOCC2)C3═NC(═NC═ | | | | | | |
| | C3)NC4═CC═C(C═C4) | | | | | | |
| | N5CCOCC5 | | | | | | |
| TBB | BrC1═C(Br)C(═C2N═N | 90341691 | | 2 | 32.64 | 33.7 | 28.55 |
| | [NH]C2═C1Br)Br | | | | | | |
| Thiostrepton | CCC(C)C1NC2\C═C/C3═ | | 1393-48-2 | 2 | 14.69 | 30.2 | 28.41 |
| from | C(N═C(C═C3C(C)O)C(═O) | | | | | | |
| Streptomyces | OC(C)C4NC(═O)C5═ | | | | | | |
| azureus | CSC(═N5)C(NC(═O)C\6 | | | | | | |
| | CSC(═N6)\C(NC(═O)C | | | | | | |
| | (NC(═O)C7═CSC(═N7)C8 | | | | | | |
| | (CCC(═N/C8C9═CSC4═ | | | | | | |
| | N9)\C%10═NC(═CS%10) | | | | | | |
| | C(═O)NC(═C)C(═O)NC | | | | | | |
| | (═C)C(N)═O)NC(═O)C(C) | | | | | | |
| | NC(O)C(C)NC(O)C( | | | | | | |
| | C)NC1═O)C(C)O)═C\C) | | | | | | |
| | C(C)(O)C(C)O)C2O | | | | | | |
| Pyridone 6 | CC(C)(C)C1═NC2═C | 174006716 | 457081-03-7 | 4 | | 0 | 28.9 |
| | ([NH]1)C3═CC═C(F)C═ | | | | | | |
| | C3C4═C2\C═C/NC4═O | | | | | | |
| tryptoline | C1CC2═C(CN1)[NH] | | | −1 | 9E−04 | −33.1 | 27.93 |
| | C3═CC═CC═C23 | | | | | | |
| Pyrintegrin | OC1═CC2═C(C═C1)N | | 1228445-38-2 | 2 | 3.289 | 31 | 27.85 |
| | (CCC2)C3═NC(═NC═C3)N | | | | | | |
| | C4═CC═C(C═C4)[S+] | | | | | | |
| | ([O−])(═O)NCC5CC5 | | | | | | |
| CEP-33779 | CN1CCN(CC1)C2═CC═ | 174006672 | 1346168-57-7 | 1 | 5.899 | 25.5 | 27.79 |
| | CC(═C2)NC3═N[N]4C═ | | | | | | |
| | CC═C(C5═CC═C(C═C5) | | | | | | |
| | [S+](C)([O−])═O)C4═N3 | | | | | | |
| Torin-1 | CCC(═O)N1CCN(CC1)C | 174006448 | 1222998-36-8 | 2 | 10.49 | 31.2 | 27.66 |
| | 2═C(C═C(C═C2)N3C(═O)\ | | | | | | |
| | C═C/C4═C3C5═CC(═C | | | | | | |
| | C═C5N═C4)C6═CN═C7 | | | | | | |
| | C═CC═CC7═C6)C(F)(F)F | | | | | | |
| URMC-099 | CN1CCN(CC1)CC2═CC═ | | 1229582-33-5 | 2 | 5.212 | 33.6 | 27.06 |
| | C(C═C2)C3═CC4═C | | | | | | |
| | ([NH]C═C4C5═CC═C6[NH] | | | | | | |
| | C═CC6═C5)N═C3 | | | | | | |
| NIDA-41020 | COC1═CC═C(C═C1)C2═ | | 502486-89-7 | 2 | 23.28 | 30.5 | 26.91 |
| | C(C)C(═N[N]2C3═CC═C | | | | | | |
| | (C1)C═C3C1)C(═O) | | | | | | |
| | NN4CCCCC4 | | | | | | |
| NCGC00385284-01 | CC(/CC[C@@H]1C(═C) | | | −1 | 0.001 | −31.2 | 26.38 |
| | CC[C@@H]2[C@@](C) | | | | | | |
| | (CO[C@@H]3O[C@H] | | | | | | |
| | (CO)[C@@H](O)[C@H](O) | | | | | | |
| | [C@H]3O)[C@H](O)CC | | | | | | |
| | [C@@H]12C)═C\CO | | | | | | |
| | [C@@H]4O[C@H](CO) | | | | | | |
| | [C@H](O)[C@H](O) | | | | | | |
| | [C@H]4O | | | | | | |
| podosporin A | C/C═C/C(═O)[C@@H] | | | 4 | | 0 | 26.17 |
| | (C)/C1═C/C(═O)/C2═C(/O | | | | | | |
| | [C@@]34CC[C@H](O)C | | | | | | |
| | (C)(C)[C@@H]3CC[C@H] | | | | | | |
| | (C)[C@@]4(C)C2)C1═O | | | | | | |
| H-89 | [O−][S+](═O) | 124892364 | 127243-85-0 | 4 | 1.866 | 16.9 | 25.95 |
| | (NCCNC\C═C\C1═ | | | | | | |
| | CC═C(Br)C═C1)C2═ | | | | | | |
| | CC═CC3═CN═CC═C23 | | | | | | |
| ML192 | CC1═NC2═C(C3═C(CCC | | 460331-61-7 | 4 | 0.521 | 13.5 | 25.23 |
| | C3)S2)C(═N1)N4CCN(C | | | | | | |
| | C4)C(═O)C5═CC═CO5 | | | | | | |
| NCGC00380931-01 | CC1(CCOC2OC(CO)C(O) | | | 2 | 11.43 | 28 | 24.96 |
| | C(O)C2O)CCC34OC3(C1) | | | | | | |
| | C(O)CC5C(C)(CCCC4 | | | | | | |
| | 5C)C(O)═O | | | | | | |
| GLYCYRRHIZIC | CC1(C)[C@H](CC[C@@] | | 1405-86-3 | 2 | 14.69 | 26 | 23.95 |
| ACID. | 2(C)C1CC[C@@]3(C) | | | | | | |
| AMMONIUM | C\4CC[C@@]5(C)CC | | | | | | |
| SALT | [C@@](C)(C[C@H] | | | | | | |
| | 5C4═C/C(═O)C23)C([O−])═ | | | | | | |
| | O)O[C@@H]6O[C@@H] | | | | | | |
| | ([C@@H](O[C@@H] | | | | | | |
| | 7O[C@@H]([C@@H] | | | | | | |
| | (O)[C@H](O)[C@H]7O) | | | | | | |
| | C([O−])═O) | | | | | | |
| | [C@H](O)[C@H]6O) | | | | | | |
| | C([O−])═O | | | | | | |
| NCGC00384655-01 | CC(═O)O[C@H]1C(═O) | | | 2 | 1.739 | 29.1 | 23.91 |
| | [C@]2(C)C(O)C[C@H]3 | | | | | | |
| | OC[C@@]3(OC(C)═O) | | | | | | |
| | [C@H]2[C@H](OC(═O)C | | | | | | |
| | 4═CC═CC═C4)[C@]5(O) | | | | | | |
| | C[C@H](O)C(═C1C5 | | | | | | |
| | (C)C)C | | | | | | |
| CEP-28122 | COC1═C(NC2═NC(═C | | 1354545-57-5 | 1 | 0.585 | 23.5 | 23.83 |
| | (C1)C═N2)N[C@@H]3 | | | | | | |
| | [C@@H]\4C[C@@H](\C═C4) | | | | | | |
| | [C@@H]3C(N)═O)C═C | | | | | | |
| | C5═C1CC[C@H](CC5)N | | | | | | |
| | 6CCOCC6 | | | | | | |
| NCGC00385739-01 | C[C@@H]1O[C@@H](O | | | 2 | 3.126 | 24.5 | 23.02 |
| | [C@@H]2[C@@H](O) | | | | | | |
| | [C@@H](O)[C@@H](CO) | | | | | | |
| | O[C@H]2O[C@@H]3 | | | | | | |
| | [C@@H](O)[C@H](O) | | | | | | |
| | [C@H](O[C@H]3O[C@H] | | | | | | |
| | 4CC[C@@]5(C)C(CC[C@] | | | | | | |
| | 6(C)C5C\C═C/7[C@H] | | | | | | |
| | 8CC(C)(C[C@@H](O) | | | | | | |
| | [C@]8(C)CC[C@@]6 | | | | | | |
| | 7C)C(O)═O)[C@@]4(C) | | | | | | |
| | CO)C(O)═O)[C@H](O) | | | | | | |
| | [C@H](O)[C@H]1O | | | | | | |
| AF38469 | CC1═NC(═CC═C1)NC | | 1531634-31-7 | 2 | 5.212 | 29.6 | 22.96 |
| | (═O)C2═CC═C(C═ | | | | | | |
| | C2C(O)═O)C(F)(F)F | | | | | | |
| (+)-Pagoclone | CC(C)CCC(═O)CC1N | | 133737-32-3 | 4 | | 0 | 22.94 |
| | (C(═O)C2═CC═CC═C12) | | | | | | |
| | C3═CC═C4C═CC | | | | | | |
| | (═NC4═N3)C1 | | | | | | |
| NCGC00384860-01 | COC1═CC═C2[C@@H]3 | | | 2 | 18.49 | 29.1 | 22.82 |
| | OC4═C(C═CC(═C4)O) | | | | | | |
| | [C@@H]3COC2═C1O | | | | | | |
| NCGC00386932-01 | COC(═O)C1═C(N═CC(═ | | | −1 | 0.004 | −26.5 | 22.59 |
| | C1)Br)N2CCC(NC3CCC | | | | | | |
| | CC3)C(O)C2 | | | | | | |
| Doxofylline | CN1C(═O)N(C)C2═C | | 69975-86-6 | 4 | 0.521 | 12.1 | 22.52 |
| | [N](CC3OCCO3)C═ | | | | | | |
| | N2)C1═O | | | | | | |
| ALLOXAN | O═C1NC(═O)C(═O) | | 11/3/2244 | 2 | 5.212 | 27.2 | 22.45 |
| | C(═O)N1 | | | | | | |
| OXYPHENBU | CCCCC1C(═O)N(N(C1═ | | 7081-38-1, | 2 | 23.28 | 25.7 | 22.37 |
| TAZONE | O)C2═CC═C(O)C═C2)C | | 129-20-4 | | | | |
| | 3═CC═CC═C3 | | [anhydrous] | | | | |
| URSOLIC | C[C@@H]1CC[C@@]2 | | 77-52-1 | 4 | 0.521 | 11.7 | 22.27 |
| ACID | (CC[C@]3(C)\C(═C/CC4 | | | | | | |
| | [C@@]5(C)CC[C@H](O) | | | | | | |
| | C(C)(C)C5CC[C@@]34 | | | | | | |
| | C)C2[C@H]1C)C(O)═O | | | | | | |
| NCGC00381003-01 | C[C@@H]1O[C@@H] | | | 2 | 2.483 | 24.5 | 22.06 |
| | (OC2C(O)C(O)C(CO)OC2 | | | | | | |
| | O[C@@H]3[C@@H](O) | | | | | | |
| | [C@H](O)[C@H](O[C@H] | | | | | | |
| | 3O[C@H]4CC[C@@] | | | | | | |
| | 5(C)C(CC[C@]6(C)C5C\ | | | | | | |
| | C═C/7C8CC(C)(C)CC(O) | | | | | | |
| | [C@]8(C)CC[C@@]67C) | | | | | | |
| | C4(C)C)C(O)═O)[C@H] | | | | | | |
| | (O)[C@H](O)[C@H]1O | | | | | | |
| BMS-863233 | C1C1═CC2═C(OC3═C2N | | 1169562-71-3 | 2 | 26.12 | 28.3 | 22.03 |
| (XL-413) | C(═N\C3═O)/[C@@H]4 | | | | | | |
| | CCCN4)C═C1 | | | | | | |
| Dabrafenib | CC(C)(C)C1═NC(═C(S1) | 174006404 | 405554-55-4 | 2 | 14.82 | 21.7 | 21.96 |
| | C2═NC(═NC═C2)N)C3═ | | | | | | |
| | C(F)C(═CC═C3)N[S+]([O−]) | | | | | | |
| | (═O)C4═C(F)C═CC═C4F | | | | | | |
| NCGC00168845-02 | OCC(═C)C(═O)O[C@H] | | | 2 | 3.613 | 27.1 | 21.8 |
| | 1CC(═C)[C@@H]2C | | | | | | |
| | [C@H](O)C(═C)[C@@H]2 | | | | | | |
| | [C@H]3OC(═O)C(═C) | | | | | | |
| | [C@H]13 | | | | | | |
| 19- | C[C@]12CC(O)C3[C@@H] | | | 4 | 0.004 | −11.5 | 21.79 |
| hydroxysarment | (CC[C@@H]4C[C@@H] | | | | | | |
| ogenin | (O)CC[C@]34CO) | | | | | | |
| | [C@@]1(O)CC[C@@H] | | | | | | |
| | 2\C5═C\C(═O)OC5 | 90341808 | | 2 | 20.6 | 23.7 | 21.78 |
| CCG-2046 | CCCC1(C)C(C#N)(C#N) | | | | | | |
| | C1(C#N)C#N | | | | | | |
| Tipranavir | CCC[C@@]1(CCC2═CC═ | | 174484-41-4 | 2 | 26.12 | 26.5 | 21.75 |
| | CC═C2)CC(═C([C@H] | | | | | | |
| | (CC)C3═CC═CC(═C3)N | | | | | | |
| | [S+]([O−])(═O) | | | | | | |
| | C4═CC═C(C═N4) | | | | | | |
| | C(F)(F)F)/C(═O)O1)\O | | | | | | |
| Sitaxenian | CC1═C(CC(═O)C2═C(C═ | | 210421-74-2 | 2 | 26.12 | 27.1 | 21.69 |
| sodium | CS2)[S](═O)(═O)[N−] | | | | | | |
| | C3═C(C1)C(═NO3)C)C═ | | | | | | |
| | C4OCOC4═C1 | | | | | | |
| VU 0285683 | [F]C1═CC(═CC(═C1)C2═ | | [327056-22-4] | 4 | | 0 | 21.28 |
| | NC(═NO2)C3═NC═ | | | | | | |
| | CC═C3)[C]#[N] | | | | | | |
| CHIR-124 | C1C1═CC\2═C(NC(═O)\ | 174007007 | 405168-59-4 | 4 | 0.235 | 12.4 | 21.27 |
| | C(═C2N[C@@H]3CN4C | | | | | | |
| | CC3CC4)C5═NC6═C | | | | | | |
| | ([NH]5)C═CC═C6)C═C1 | | | | | | |
| Tioguanine | NC/1═N/C2═C([NH] | 174007001 | 15442-7 | 4 | | 0 | 21.27 |
| | C═N2)C(═S)N1 | | | | | | |
| DIHYDROCEL | CC1═C2C\C═C\3[C@@] | | | 2 | 14.69 | 26.1 | 21.22 |
| ASTROL | (C)(CC[C@@]4(C)[C@H] | | | | | | |
| | 5C[C@@](C)(CC[C@] | | | | | | |
| | 5(C)CC[C@]34C)C(O)═ | | | | | | |
| CP-724714 | COCC(═O)NC/C═C/C1═ | | 383432-38-0 | 2 | 20.75 | 23.5 | 21.17 |
| | CC═C2N═CN═C(NC3═C | | | | | | |
| | C═C(OC4═CN═C(C)C═ | | | | | | |
| | C4)C(═C3)C)C2═C1 | | | | | | |
| Edaravone | C\C1═N\N(C(═O)C1) | | 89-25-8 | 4 | 0.521 | 11 | 20.97 |
| | C2═CC═CC═C2 | | | | | | |
| Gynostemma | CC(C)═CCC[C@](C)(O | | 80321-63-7 | 4 | 0.256 | 11 | 20.39 |
| Extract | [C@@H]1O[C@H](CO | | | | | | |
| | [C@@H]2OC[C@@H](O) | | | | | | |
| | [C@H](O)[C@H]2O) | | | | | | |
| | [C@@H](O)[C@H](O)[C@H] | | | | | | |
| | 1O)[C@H]3CC[C@]4(C) | | | | | | |
| | [C@@H]3[C@H](O)C | | | | | | |
| | [C@@H]5[C@@]6(C)CC | | | | | | |
| | [C@H](O[C@@H]7O | | | | | | |
| | [C@H](CO)[C@@H](O) | | | | | | |
| | [C@H](O)[C@H]7O)C(C) | | | | | | |
| | (C)[C@@H]6CC | | | | | | |
| | [C@@]45C | | | | | | |
| Oleanolic acid | CC1(C)C[C@H](C2CC | | 508-02-1 | 4 | 0.37 | −10.7 | 20.26 |
| | [C@]3(C)\C(═C/C[C@@H] | | | | | | |
| | 4[C@@]5(C)CC[C@H] | | | | | | |
| | (O)C(C)(C)[C@@H]5C | | | | | | |
| | C[C@@]34C)1C[C@@H]2 | | | | | | |
| | C1)C(O)═O | | | | | | |

¹Compound notation in a “simplified molecular-input line-entry system” (SMILES), a line notation for describing the structure of chemical species using short ASCII strings
²Compound identifier in PubChem database maintained by the National Center for Biotechnology Information (NCBI), a component of the National Library of Medicine, which is part of the United States National Institutes of Health (NIH).
³A unique numerical identifier assigned by the Chemical Abstracts Service (CAS).
⁴Curve class based on the classification provided in Huang et al. Environ. Health Perspeet. 119:1142-1148 (2011).
⁵The concentration at 50% efficacy. AC₅₀ is not available for curve classes (CCs) 4 and 5.
⁶The activity range between predicted activities at zero and infinite doses, fitted using Hill equation. Efficacy is not available for CCs 4 and 5.
⁷Maximum activity value from all tested concentrations.

TABLE 2
Compounds screed for potential synergy with Chroman 1 and Emricasan

| Compound | SMILES¹ | Concentration (μM) |
|---|---|---|
| Isotretinoin | CC/1═C(\C═C\C(C)═C\C═C\C(C)═C/C(O)═O)C(C)(C)CCC1 | 0.707945784 |
| Pranazepide | Fc6ccccc6C3═N[C@H](N C(═O)c1cc2ccccc2n1)C(═O)N5CCc4cccc3c45 | 2.089296131 |
| Ergosterol | CC(C)[c@@H](C)\C═C\[C@@H](C)[C@H]2\C3═C\C═C4\C[C@@H](O)CC | 9.54992586 |
| | [C@]4(C)[C@H]3CC[C@]12C | |
| AC-261066 | CCCCOCCOC1═C(C)SC(═N1)C2═CC═C(C(O)═O)C(═C2)F | 2.089296131 |
| Methylnaltrexone | C[N@+]1(CC[C@@]23[C@H]4OC5═C(O)C═CC(═C25)C[C@@H]1 | 17.7827941 |
| (Bromide) | [C@]3(O)CCC4═O)CC6CC6 | |
| MLS002707279-02 | CC(═O)C1(C)CCC2C3C═C(C)C4═CC(═O)CCC4(C)C3CCC12C | 0.707945784 |
| Vadadustat | OC(═O)CNC(═O)C1═NC═C(C(C═1O)C2═CC═CC(═C2)C1 | 0.234422882 |
| trans-ISRIB | C1C1═CC═C(OCC(═O)N[C@H]2CC[C@@H](CC2)NC(═O)COC3═CC═C | 0.707945784 |
| | (C1)C═C3)C═C1 | |
| CT-98014 | NC1═NC(═CC═C1[N+](O−])═O)NCCNC2═NC═C([N]3C═CN═C3)C(═N2) | 0.316227766 |
| | C4═CC═C(C1)C═C4C1 | |
| KRIBB11 | CNC1═NC(═C(C═C1)[N+]([O−])═O)NC2═CC═C3[NH]N═CC3═C2 | 2.089296131 |
| NCGC00386849-01 | COC1═C(C═CC═C1)C2═CC═C(C═C2)C3═C[N](N═N3)C(═O) | 0.234422882 |
| | N4CCCCC4CC5═CC═CC═C5 | |
| WAY 267464 | C[N]1N═CC2═C1NC3═C(C═CC═C3)N(C2)C(═O)C4═CC═C(CNC(═O) | 0.025703958 |
| dihydrochloride | N5CCN(CC5)CC6═CC(═CCC(═C4)C(═C6)O)O) | |
| PTC- 124 | OC(═O)C1═CC═CC(═C1)C2═NOC(═N2)C3═CC═CC═C3F | 0.02630268 |
| PF 05081090 | COC1═CC═C(C(═C(C(═C1)F)C\2═C\C(═O)N (CC[C@](C)(C(═O)NO)[S+] | 0.231739465 |
| | (C)(O−])═O])═O)\C═C2 | |
| Chlortetracycline | CN(C)[C@H]\1[C@@H]2CC3\C(═C(\O)[C@]2(O)C(═O)\C(═C1O)C(N)═O)C(═O) | 0.234422882 |
| HCl | C4═C(C(═CC═C4O)C1)[C@@]3(C)O | |
| Ceftiofur HCl | CO\N═C(\C(═O)N[C@H]1[C@H]2SC\C(═C(N2C1═O)C(O)═O)CSC(═O) | 0.707945784 |
| | C3═CC═CO3)C4═CSC(═N4)N | |
| NCGC00386840-01 | C[C@@H](N(C1CCCCC1)C(═O)C[N]2N═NC(═N2)C3═CC4═C(OCO4)C═C3)C | 0.707945784 |
| | (═O)NC5CCCC5 | |
| NCGC00385453-01 | COC1CC(OC(C)C1OC2CC(OC)C(OC3OC(C)C(OC4OC(CO)C(O)C(O)C4O)C(OC) | 0.006456542 |
| | C3O)C(C)O2)OC5CCC6(C)C7CC(OC(═O)/C(C)═C\C)C8(C)C(CCC8(O) | |
| | C7C/C═C6/C5)C(C)═O | |
| Zolmitriptan | CN(C)CCC1═C[NH]C2═CC═C(C[C@H]3COC(═O)N3)C═C12 | 0.1 |
| Cyclosporine A | CCC1NC(═O)C(C(O)C(C)C\C═C\C)N(C)C(═O)C(C(C)C)N(C)C(═O)C(CC(C)C) | 0.02630268 |
| | N(C)C(═O)C(CC(C)C)N(C)C(═O)C(C)NC(═O)C(C)NC(═O)C(CC(C)C)N(C)C(═O) | |
| | C(NC(═O)C(CC(C)C)N(C)C(═O)CN(C)C1═O)C(C)C | |
| NCGC00169917-02 | COC1═CC2═C(C(═C1)O)C(=O)C3(O2)C(C)CC(=O)/C═C3/OC | 0.695024318 |
| PD-166793 | CC(C)[C@H](C[S+]([O−])(═O)C1═CC═C(C═C1)C2═CC═C(Br)C═C2)C(O)═O | 0.234422882 |
| SB-206553 | C[N]1C═CC2═CC3═C(CCN3C(═O)NC4═CC═CN═C4)C═C12 | 1.77827941 |
| Dipraglurant | FC1═C[N]2C═C(CCC#CC3═NC═CC═C3)N═C2C═C1 | 0.234422882 |
| INF 4E | CCOC(═O)C(═C)C(O)C1═C(C1)C═CC═C1 | 0.707945784 |
| IWP-3 | CC1═CC═C2N═C(NC(═O)CS\C3═N\C4═C(SCC4)/C(═O)N3C5═CO═C(F)C═C5) | 0.316227766 |
| | SC2═C1 | |
| NCGC00380777-01 | CCC(C)(NC(═O)C(CCC(N)═O)NC(═O)C1CC(O)1C(═O)C(C)(C)NC(═O)C(C)(C)NC | 0.120226443 |
| | (═O)C(CC(C)C)NC(═O)CNC(═O)C(NC(═O)C(C)(C)NC(═O)C(C)(C)NC(═O)C(C)(C) | |
| | NC(═O)C(CC2═CC═CC═C2)NC(C)═O)C(C)C(C(═O)N3CC(O)CC3C(═O)NC(C)(C) | |
| | C(═O)NC(CO)CC4═CC═CC═C4 | |
| Xestospongin C | C1CCC[C@@H]2CCCN3CC[C@@H](CCCCCC[C@H]4CCCN5CC[C@@H](CC1)O | 0.01 |
| | [C@@H]45)O[C@@H]23 | |
| Necrosulfonamide | COC1═C(N[S+]([O−])(═O)C2═CC═C(NC(═O)\C═C\C3═CC═C(S3)[N+]([O−])═O) | 0.077624712 |
| | C═C2)N═CC═N1 | |
| Cathepsin Inhibitor 1 | C[N]1N═C(C═C1C(═O)N[C@@H](CC2═CC═CC(═C2)C1)C(═O)NCC#N)C(C)(C)C | 0.707945784 |
| N6-p- | OC[C@H]1O[C@H]([C@H](O)[C@@H]1O)[N]2C═NC3═C(NC4═CC═C(C═C4)[S] | 1.77827941 |
| Sulfophenyladenosine | ([O−])(═O)═O)N═CN═C23 | |
| sodium salt | | |
| Radotinib(IY-5511) | CC1═C[N](C═N1)C2═CC(═CC(═C2)\N═C(/O)C3═CC(═C(C)C═C3)NC4═NC═CC | 0.077624712 |
| | (═N4)C5═CN═CC═N5)C(F)(F)F | |
| PF 750 | O═C(NC1═CC═CC═C1)N2CCC(CC2)CC3═CC4═CC═CC═C4N═C3 | 0.695024318 |
| TC LPA5 4 | COC1═CC(═CC═C1)[N]2N═C(C═C2C3═CC(═C(C═C3)C4CCCCC4)C1)C(O)═O | 2.089296131 |
| Honokiol | OC1═CC═C(C═C1CC═C)C2═CC(═CC═C2O)CC═C | 0.1 |
| Pifithrin-u | N[S+]([O−])(═O)C#CC1═CC═CC═C1 | 0.077090347 |

¹Compound notation in a “simplified molecular-input line-entry system” (SMILES), a line notation for describing the structure of chemical species using short ASCII strings

1. A composition comprising Chroman 1 and/or a derivative thereof and Emricasan and/or a derivative thereof.

2. The composition of claim 1 further comprising one or both of trans-ISRIB and polyamines.

3. The composition of claim 1, wherein the composition is formulated to provide a concentration of Chroman 1 and/or the derivative thereof of about 4 nM to about 80 μM, 4 nM to about 40 μM, about 10 nM to about 20 μM, about 20 nM to about 10 μM or about 30 nM to about 500 nM, when incorporated into a medium.

4. The composition of claim 1, wherein the composition is formulated to provide a concentration of Emricasan and/or the derivative thereof of about 100 nM to about 80 μM, about 100 nM to about 40 μM, about 200 nM to about 30 μM, about 300 nM to about 20 μM, when incorporated into the medium.

5. The composition of claim 2, wherein the composition is formulated to provide a concentration of trans-ISRIB of about 50 nM to about 80 μM, about 50 nM to about 40 μM, about 50 nM to about 20 μM, about 50 nM to about 10 μM, about 50 nM to about 6.25 μM, about 100 nM to about 6.25 μM, or about 200 nM to about 6.25 μM, when incorporated into the medium.

6. The composition of claim 2, wherein the composition comprises polyamines comprising spermine and spermidine, and wherein the composition is formulated to provide a concentration of spermine of about 0.5 nM to 1 mM, and/or a concentration of spermidine of about 0.5 nM to 1 mM, when incorporated into the medium.

7. The composition of claim 6, wherein the polyamines further comprise putrescine, and wherein the composition is formulated to provide a concentration of putrescine of about 0.5 nM to 1 mM, when incorporated into the medium.

8. A medium composition comprising components configured to support at least one mammalian cell in vitro or ex vivo and Chroman 1 and/or a derivative thereof.

9. The medium composition of claim 8, comprising Chroman 1 and/or the derivative thereof at an effective concentration of about 4 nM to 80 μM, 4 nM to 40 μM, 10 nM to 20 μM, 20 nM to 10 μM or 30 nM to 500 nM, wherein the effective concentration of Chroman 1 and/or the derivative thereof is a concentration of Chroman 1 and/or the derivative thereof in a working medium to be used without further dilution.

10. The medium composition of claim 8, further comprising Emricasan and/or a derivative thereof.

11. The medium composition of claim 8, comprising Emricasan at an effective concentration of about 100 nM to 80 μM, 100 nM to 40 μM, 200 nM to 300 μM, 300 nM to 20 μM, wherein the effective concentration of Emricasan and/or the derivative thereof is a concentration Emricasan and/or the derivative in the working medium to be used without further dilution.

12. The medium composition of claim 8, further comprising trans-ISRIB.

13. The medium composition of claim 8, comprising trans-ISRIB at an effective concentration of about 50 nM to about 80 μM, about 50 nM to about 40 μM, about 50 nM to about 20 μM, about 50 nM to about 10 μM, about 50 nM to 6.25 μM, 100 nM to 6.25 μM, or 200 nM to 6.25 6.25 μM, wherein the effective concentration of trans-ISRIB is trans-ISRIB concentration in the working medium to be used without further dilution.

14. The medium composition of claim 8, further comprising polyamines comprising spermine and spermidine.

15. The medium composition of claim 8, wherein the medium composition comprises an effective concentration of spermine of about 0.5 nM to 1 mM, and/or an effective concentration of spermidine of about 0.5 nM to 1 mM, and wherein the effective concentration of each of spermine and spermidine is a concentration in the working medium to be used without further dilution.

16. The medium of claim 14, wherein the polyamines further comprise putrescine.

17. The medium of claim 16, wherein the medium composition comprises an effective concentration of putrescine of about 0.5 nM to 1 mM, wherein the effective concentration of putrescine is a concentration in the working medium to be used without further dilution.

18. The medium composition of claim 8, wherein the components configured to support the at least one mammalian cell in vitro or ex vivo comprise one or more of a buffer, an inorganic salt, essential amino acids, a carbohydrate, fatty acids, lipids, vitamins and trace elements.

19-25. (canceled)

26. A kit comprising the composition of claim 1 and medium components configured to support at least one mammalian cell in vitro or ex vivo.

27. The kit of claim 26, further comprising at least one of a culture vessel, a support or a scaffold for growth of the at least one mammalian cell in vitro or ex vivo.

28-56. (canceled)
